(19) United States
(12) Patent Application Publication
(10) Pub. No.: US 2025/0017634 A1
(43) Pub. Date: Jan. 16, 2025

(54) IMPLANTS, INSTRUMENTS, AND METHODS OF USE

(71) Applicant: Paragon 28, Inc., Englewood, CO (US)

(72) Inventors: Michael SCHMIDT, Greenwood Village, CO (US); Richard David HUNT, Arvada, CO (US); Lucas Charles MCMAHAN, Lone Tree, CO (US)

(73) Assignee: Paragon 28, Inc., Englewood, CO (US)

(21) Appl. No.: 18/896,358

(22) Filed: Sep. 25, 2024

Related U.S. Application Data
(63) Continuation of application No. PCT/US2023/064989, filed on Mar. 27, 2023.
(60) Provisional application No. 63/269,934, filed on Mar. 25, 2022.
(60) Provisional application No. 63/480,416, filed on Jan. 18, 2023.

(51) Int. Cl. A61B 17/72; A61B 17/86

(52) U.S. Cl. CPC A61B 17/7291; A61B 17/8605; A61B 17/151

(57) ABSTRACT

An implant having a proximal portion, a distal portion and a central portion positioned between the proximal portion and the distal portion. The implant further includes a longitudinal axis with the proximal portion, the distal portion and the central portion being centered along the longitudinal axis. An implant system that includes an implant having a proximal portion including an opening, a distal portion including a threading, a tip, and a tapping feature at the tip, and a central portion having at least one flat on an outer surface The central portion is disposed between the proximal and distal portions. The implant system also includes an insertion instrument configured to be releasably coupled with the implant.

### CROSS REFERENCE TO RELATED APPLICATIONS

This application is a continuation of International Application No. PCT/US2023/064989 filed on Mar. 27, 2023 and entitled “Implants, Instruments, And Methods Of Use,” which claims priority benefit under 35 U.S.C. § 119(e) to U.S. Provisional Patent Application No. 63/269,934 filed Mar. 25, 2022, and entitled “Implants, Instruments, And Methods Of Use,” and U.S. Provisional Patent Application No. 63/480,416 filed Jan. 18, 2023, and entitled “Implants, Instruments, And Methods Of Use,” which are hereby incorporated herein by reference in their entireties.

### TECHNICAL FIELD

The present disclosure relates to surgical implants, instruments, systems, and methods of use to be implemented in surgical procedures. The present disclosure relates to podiatric and orthopedic surgical implants, instruments, systems, and methodology to be implemented in various procedures of the foot and/or ankle, for example various intramedullary procedures. More specifically, but not exclusively, the present disclosure relates to surgical implants, instruments, systems, and methods to be implemented in performing intramedullary procedures of the metatarsals and phalanges.

### BACKGROUND OF THE INVENTION

Many currently available surgical implants, instruments, and systems, as well as methodology, do not completely address the needs of patients. Additionally, many currently available surgical implants, instruments, systems, and methodology fail to account for properties of joint anatomy and accordingly can decrease favorability of the outcome for the patient.

### SUMMARY OF THE INVENTION

The present disclosure is directed toward surgical implant for implementation in conjunction with instruments, and methods directed to the repair of bones, arthrodesis and other similar procedures.

A first aspect of the present disclosure is an implant having a proximal portion, a distal portion and a central portion positioned between the proximal portion and the distal portion. The implant further includes a longitudinal axis with the proximal portion, the distal portion and the central portion being centered along the longitudinal axis.

A second aspect of the present disclosure is an implant system. The implant system includes an implant having a proximal portion including an opening, a distal portion including a threading, a tip, and a tapping feature at the tip, and a central portion having at least one flat on an outer surface, where the central portion is disposed between the proximal and distal portions. The implant system also includes an insertion instrument configured to releasably couple with the implant.

### BRIEF DESCRIPTION OF THE DRAWINGS

The accompanying drawings, which are incorporated in and constitute a part of the specification, illustrate embodiments of the inventions and together with the detailed description herein, serve to explain the principles of the inventions. It is emphasized that, in accordance with the standard practice in the industry, various features may or may not be drawn to scale. In fact, the dimensions of the various features may be arbitrarily increased or reduced for clarity of discussion. The drawings are only for purposes of illustrating embodiments of inventions of the disclosure and are not to be construed as limiting the inventions.

FIG. 1 is a perspective view of an exemplary implant, in accordance with the present disclosure;

FIG. 2 is a side view of the exemplary implant of FIG. 1, in accordance with the present disclosure;

FIG. 3 is a front perspective view of the exemplary implant of FIG. 1, in accordance with the present disclosure;

FIG. 4 is a rear perspective view of the exemplary implant of FIG. 1, in accordance with the present disclosure;

FIG. 5 is a rear view of the exemplary implant of FIG. 1, in accordance with the present disclosure;

FIG. 6 is a side view of an exemplary implant, in accordance with the present disclosure;

FIG. 7 is a side perspective view of the exemplary implant of FIG. 6, in accordance with the present disclosure;

FIG. 8 is a front perspective view of the exemplary implant of FIG. 6, in accordance with the present disclosure;

FIG. 9 is a rear perspective view of the exemplary implant of FIG. 6, in accordance with the present disclosure;

FIG. 10 is an alternate rear perspective view of the exemplary implant of FIG. 6, in accordance with the present disclosure;

FIG. 11 is a rear view of the exemplary implant of FIG. 6, in accordance with the present disclosure;

FIG. 12 is a side perspective view of an exemplary implant, in accordance with the present disclosure;

FIG. 13 is a side view of the exemplary implant of FIG. 12, in accordance with the present disclosure;

FIG. 14 is a front perspective view of the exemplary implant of FIG. 12, in accordance with the present disclosure;

FIG. 15 is a rear perspective view of the exemplary implant of FIG. 12, in accordance with the present disclosure;

FIG. 16 is an alternate rear perspective view of the exemplary implant of FIG. 12, in accordance with the present disclosure;

FIG. 17 is a rear view of the exemplary implant of FIG. 12, in accordance with the present disclosure;

FIG. 18 is a side perspective view of an exemplary implant system, in accordance with the present disclosure;

FIG. 19 is an alternate side perspective view of the exemplary implant system of FIG. 18, in accordance with the present disclosure;

FIG. 20 is a top perspective view of the exemplary implant system of FIG. 18, in accordance with the present disclosure;

FIG. 21 is a bottom perspective view of the exemplary implant system of FIG. 18, in accordance with the present disclosure;

FIG. 22 is a side perspective view of an exemplary implant such as that shown in the exemplary system of FIG. 18, in accordance with the present disclosure;

FIG. 23 is a side view of an exemplary implant such as that shown in the exemplary system of FIG. 18, in accordance with the present disclosure;

FIG. 24 is a rear perspective view of an exemplary implant such as that shown in the exemplary system of FIG. 18, in accordance with the present disclosure

FIG. 25 is a side perspective view of an exemplary implant, in accordance with the present disclosure;

FIG. 26 is an alternate side view of the exemplary implant shown in FIG. 25, in accordance with the present disclosure;

FIG. 27 is a rear perspective view of the exemplary implant shown in FIG. 25, in accordance with the present disclosure;

FIG. 28 is a side view of an exemplary implant, in accordance with the present disclosure;

FIG. 29 is an alternate side view of the exemplary implant of FIG. 28, in accordance with the present disclosure;

FIG. 30 is a front perspective view of the exemplary implant of FIG. 28, in accordance with the present disclosure;

FIG. 31 is a rear view of the exemplary implant of FIG. 28, in accordance with the present disclosure;

FIG. 32 is a side perspective view of an exemplary instrument which may be implemented with the implant of FIGS. 1, 6 and/or 12, in accordance with the present disclosure;

FIG. 33 is a front perspective view of the exemplary instrument of FIG. 32 which may be implemented with the implant of FIGS. 1, 6 and/or 12 or other implants, in accordance with the present disclosure;

FIG. 34 is a top view of an exemplary instrument which may be implemented with the implant system of FIG. 18, the implant of FIGS. 22, 25, and/or 28, or other implants, in accordance with the present disclosure;

FIG. 35 is a rear perspective view of the exemplary instrument of FIG. 34 which may be implemented with the implant system of FIG. 18, the implant of FIGS. 22, 25, and/or 28, or other implants, in accordance with the present disclosure;

FIG. 36 is a side perspective view of an implant system which may be implemented with the implant of FIGS. 1, 6 and/or 12, and may also be implemented with the instrument of FIG. 32, in accordance with the present disclosure;

FIG. 37 is a front, perspective view of an implant system which may be implemented with the implant system of FIG. 18, the implant of FIGS. 22, 25 and/or 28, and may also be implemented with the instrument of FIG. 34, in accordance with the present disclosure;

FIG. 38 is a side view of an exemplary implant which may be implemented with the implant system of FIG. 18, the implant of FIGS. 22, 25 and/or 28, in accordance with the present disclosure;

FIG. 39 is a perspective view of the exemplary implant of FIG. 38 which may be implemented with the implant system of FIG. 18, the implant of FIGS. 22, 25 and/or 28, in accordance with the present disclosure;

FIG. 40 is a top view of the exemplary implant of FIG. 38 which may be implemented with the implant system of FIG. 18, the implant of FIGS. 22, 25 and/or 28, in accordance with the present disclosure;

FIG. 41 is a perspective view of an exemplary instrument which may be implemented in conjunction with one or more of the implants, systems, and/or instruments shown and described previously, in accordance with the present disclosure;

FIG. 42 is a top view of the exemplary instrument of FIG. 41 which may be implemented in conjunction with one or more of the implants, systems, and/or instruments shown and described previously, in accordance with the present disclosure;

FIG. 43 is a top perspective view of an exemplary instrument which may be implemented in conjunction with one or more of the implants, systems, and/or instruments shown and described previously, in accordance with the present disclosure;

FIG. 44 is a bottom perspective view the exemplary instrument of FIG. 43 which may be implemented in conjunction with one or more of the implants, systems, and/or instruments shown and described previously, in accordance with the present disclosure;

FIG. 45 is perspective view of an exemplary instrument which may be implemented in conjunction with one or more of the implants, systems, and/or instruments shown and described previously, in accordance with the present disclosure;

FIG. 46 is an alternate perspective view of an exemplary instrument which may be implemented in conjunction with one or more of the implants, systems, and/or instruments shown and described previously, in accordance with the present disclosure;

FIG. 47 is a perspective view of an exemplary instrument which may be implemented in conjunction with one or more of the implants, systems, and/or instruments shown and described previously, in accordance with the present disclosure;

FIG. 48 is a perspective view of an exemplary instrument which may be implemented in conjunction with one or more of the implants, systems, and/or instruments shown and described previously, in accordance with the present disclosure;

FIG. 49 is a perspective view of the exemplary instrument of FIG. 48 which may be implemented in conjunction with one or more of the implants, systems, and/or instruments shown and described previously, in accordance with the present disclosure;

FIG. 50 is a perspective view of an exemplary implant system, in accordance with the present disclosure;

FIG. 51 is a top view of a portion of the implant system of FIG. 50 positioned within a bone, in accordance with the present disclosure;

FIG. 52 is a top view of an exemplary implant, in accordance with the present disclosure;

FIG. 53 is a top view of an exemplary implant, in accordance with the present disclosure;

FIG. 54 is a top view of an exemplary implant, in accordance with the present disclosure;

FIG. 55 is a top view of an exemplary implant, in accordance with the present disclosure;

FIG. 56 is a top view of an exemplary implant, in accordance with the present disclosure;

FIG. 57 is a top view of an exemplary implant, in accordance with the present disclosure;

FIG. 58 is a top view of an exemplary implant, in accordance with the present disclosure;

FIG. 59 is a top view of an exemplary implant, in accordance with the present disclosure;

FIG. 60 is a perspective view of an exemplary instrument which may be implemented in conjunction with the implant system of FIG. 50, in accordance with the present disclosure; and

FIG. 61 is a perspective view of an exemplary instrument which may be implemented in conjunction with the implant system of FIG. 50, in accordance with the present disclosure.

### DETAILED DESCRIPTION OF THE INVENTION

In this detailed description and the following claims, the words proximal, distal, anterior or plantar, posterior or dorsal, medial, lateral, superior and inferior are defined by their standard usage for indicating a particular part or portion of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure. Further, specifically in regards to the foot, the term “dorsal” refers to the top of the foot and the term “plantar” refers the bottom of the foot.

Similarly, positions or directions may be used herein with reference to anatomical structures or surfaces. For example, as the current implants, devices, instrumentation, and methods are described herein with reference to use with the bones of the foot, the bones of the foot, ankle and lower leg may be used to describe the surfaces, positions, directions or orientations of the implants, devices, instrumentation and methods. Further, the implants, devices, instrumentation, and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, instrumentation, and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, instrumentation, and methods, and the aspects, components, features and the like thereof, described herein with respect to the right foot may be mirrored so that they likewise function with the left foot. Further, the implants, devices, instrumentation, and methods, and the aspects, components, features and the like thereof, disclosed herein are described with respect to the foot for brevity purposes, but it should be understood that the implants, devices, instrumentation, and methods may be used with other bones of the body having similar structures.

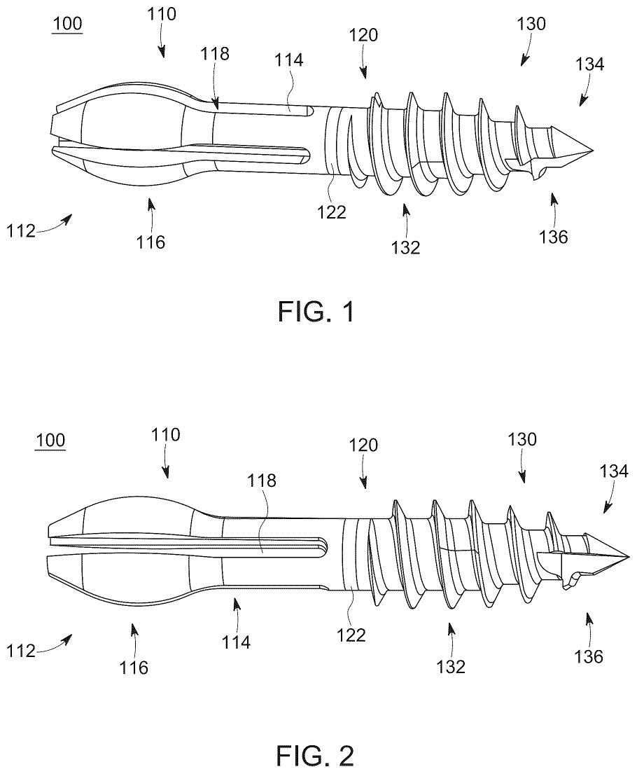

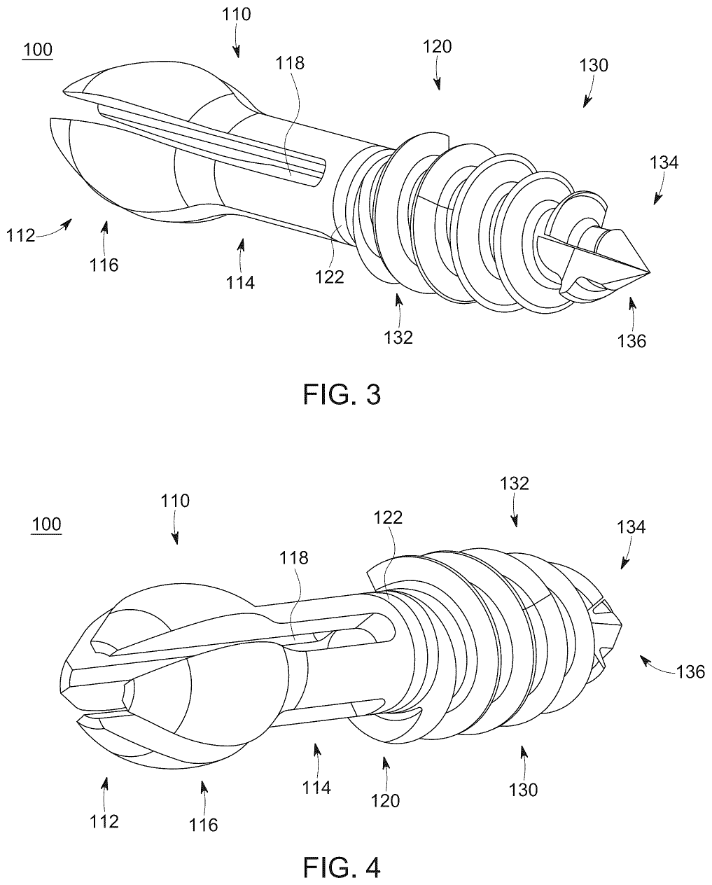

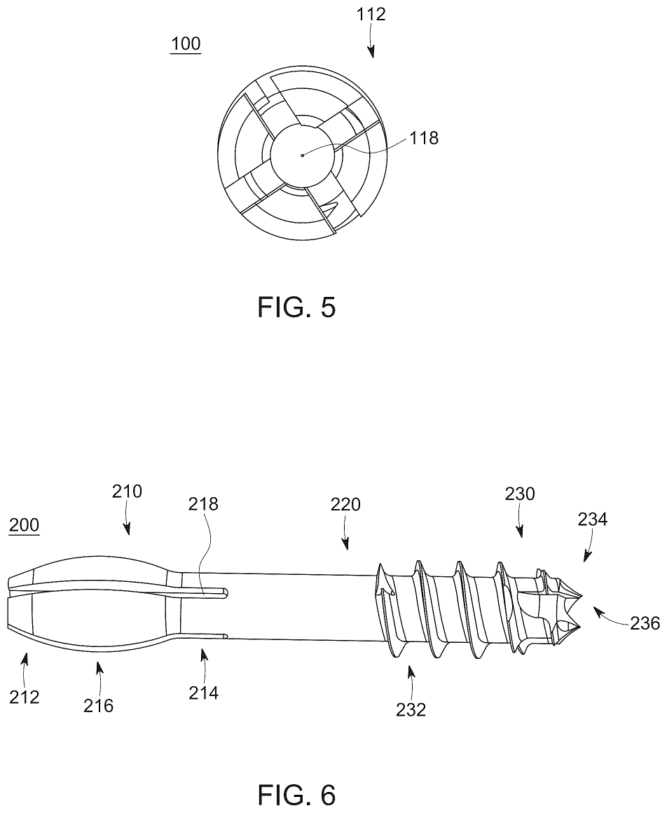

Referring now to FIGS. 1-5, an exemplary implant 100 is shown. It should be understood that, as shown and described subsequently herein, the implant 100 may be desirable in various sizes/geometries and/or other configurations in order to address various conditions, both chronic and acute, of the extremities (e.g., feet as shown in an exemplary fashion herein) as well as other portions of the anatomy. In some aspects, the implant 100 may have greater or lesser lengthwise dimensions and/or cross-sectional dimensions/geometries. For example, a portion of the implant 100 may be longer, shorter, or have greater or lesser cross-sectional dimensions than those of the implant 100 as shown with reference to FIGS. 1-5. Similarly, the implant 100 may also omit features shown and described herein and/or may include additional features.

The implant 100 is shown to include a proximal portion 110, a distal portion 130, and a central portion 120 disposed between the proximal portion 110 and the distal portion 130. The notation of proximal and distal portions 110 and 130, respectively, is arbitrary as shown in FIGS. 1-5. However, when implanted in various portions of the human anatomy, either the proximal portion 110 or the distal portion 130 may be positioned proximally (and with the other portion positioned distally) relative to the anatomy. As shown, the proximal, central, and distal portions 110, 120, and 130 are substantially centered about a common longitudinal axis. In some aspects, one or more of the proximal, central, and distal portions 110, 120, 130 may include the same and/or similar geometric shapes and/or cross-sections, for example a substantially cylindrical cross-section. As shown, each of the proximal, central, and distal portions 110, 120, and 130 are integral with one another. However, in some aspects one or more of the proximal, central, and distal portions 110, 120, and 130 may be releasably couplable with one another and/or with other components of an implant system.

The proximal portion 110 of the implant 100 is shown to extend from the central portion 120 in a direction substantially opposite from the distal portion 130. The proximal portion 110 is shown to include a plurality of projections 112 (e.g., prongs, tines, etc.) extending from the central portion 120 such that each of the plurality of projections 112 extends along an axis that is substantially parallel to the adjacent prongs in addition to being substantially parallel to the longitudinal axis of the implant 100. As shown in FIGS. 1-5, the plurality of projections 112 includes four projections, however alternate embodiments may include a lesser or greater number of projections 112. As shown, each of the plurality of projections 112 has substantially the same geometry of the other projections of the implant 100. However, in some aspects one or more projection of the plurality of projections 112 may have a different size, length, or other geometric difference from the remaining projections of the implant 100.

As shown, each of the projections 112 occupies approximately 90-degress or less of a cylinder about the longitudinal axis of the implant 100. That is to say that each of the projections 112 is positioned in a separate quadrant of such a cylinder or a circular cross-section thereof. However, as mentioned, an alternate number of projections 112 may result in alternate geometric configurations (e.g., two projections with each occupying approximately 180-degrees or less). The plurality of projections 112 are spaced from one another by a cavity 118 which, as shown, occupies the space of the longitudinal axis of the implant 100 as it extends through the proximal portion 110. The cavity 188 may include a substantially cross-shaped (e.g., a plus-sign) geometry with each projection of the cross extending outwardly from the longitudinal axis of the implant 100 and between two projections of the plurality of projections. Accordingly, the cavity 118 may have alternate geometries should the implant 100 include an alternate number of projections.

Each projection of the plurality of projections 112 is shown to include a first portion 114 (e.g., a linear portion) and a second portion 116 (e.g., a rounded portion). Each projection of the plurality of projections 112 as shown includes substantially the same geometry and, for the sake of brevity, a single projection of the plurality of projections 112 will be described in detail subsequently. The linear portion 114 is shown to be integral with the central portion 120 of the implant 100 and, as mentioned previously, extend from the central portion 120 in a direction substantially opposite that of the distal portion 130 as well as substantially parallel to the longitudinal axis of the implant 100. The linear portion 114 is shown to include three main surfaces, with two forming a substantially orthogonal angle with one another adjacent the longitudinal axis of the implant 100. The third surface is shown to be a substantially rounded, convex surface (which may correspond to a radius of the central portion 120 or other portions of the implant 100) that extends between the edges of the two orthogonal surfaces. In some aspects, each linear portion may have a geometry the same as and/or similar to a quartered section of a cylinder, and may also include a corresponding cross-sectional geometry.

The rounded portion 116 of each projection of the plurality of projections will, for the sake of brevity, also be described with reference to a single projection. The rounded portion 116 is shown to have a substantially greater lateral dimension than that of the linear portion 114 (e.g., extends further radially from the longitudinal axis of the implant 100). The outer surface of the rounded portion has a geometry similar to a fraction of the outer surface of a bulbous or elliptical (or ellipsoid) shape. With respect to the four rounded portions 116 as a whole, each includes a substantially equal surface area on the rounded portion 116 which, collectively, make up most of the outer surface of the bulbous/elliptical shape (with the portions of the cavity 118 occupying the voids). The rounded portion 116 as shown is substantially symmetrical about a line of symmetry configured perpendicular to the longitudinal axis of the implant, where said line of symmetry corresponds to a point at which the circumference/radius of the bulbous/ellipsoid shape is greatest (and said circumference/radius decreases when moving along the longitudinal axis of the implant 100 in either direction from the line of symmetry).

The plurality of projections 112 are configured such that one or more of the projections may be depressed inward (e.g., toward the longitudinal axis of the implant) so as to decrease the lateral dimension of the proximal portion 110 of the implant 100. For example, a pilot hole may be created in a portion of a bone (e.g., an intramedullary canal) with a lateral dimension less than that of the proximal portion 110 at its greatest point (the rounded portion 116) when in an un-depressed state. Accordingly, the implant 100 may be implanted in such an opening/pilot hole by depressing one or more of the plurality projections 112 (specifically, the rounded portion 116) so as to decrease the lateral dimension of the proximal portion 110 thus facilitating implantation of the proximal portion 110 within the opening/pilot hole. Once within the opening/pilot hole, the plurality of projections 112 may exhibit a resilient property, returning to the same lateral dimension as prior to depression. In some aspects, the plurality of projections 112 may return to a semi-depressed state after implantation, with a force directed radially outward from the longitudinal axis of the implant 100 facilitating implantation and retention of the proximal portion 110 of the implant 100 within the opening/pilot hole (thus preventing pull-out/increasing pull-out resistance/strength).

The central portion 120 of the implant 100 is shown to have a substantially cylindrical geometry extending from an end of the linear portion 114 of the proximal portion 110 to a proximal-most portion of the distal portion 130 of the implant 100. As shown, the central portion 120 has a lesser longitudinal dimension than that of the proximal and distal portions 110, 130. However, in alternate embodiments, the central portion 120 may include a greater longitudinal dimension than shown in FIGS. 1-5. Conversely, in some embodiments the central portion 120 may include a lesser longitudinal dimension than shown or, in some aspects, may be absent from the implant 100 (e.g., the proximal and distal portions 110, 130 are integral with one another. The central portion 120 is shown to include a marking 122 arranged on an outer surface thereof. As shown, the marking 122 spans the circumference of the central portion 120 and may function as a depth indicator to aid in insertion of the implant 100. In some aspects, the marking 122 may include multiple markings, for example multiple rings indicating various depths of insertion when the implant 100 is implanted.

The distal portion 130 is shown to include a substantially cylindrical geometry adjacent the central portion 120, with said geometry tapering toward the end of the distal portion 130 opposite the central portion 120. The distal portion is further shown to include a threading 132 extending circumferentially (e.g., as a spiral configuration) from a portion adjacent the central portion 120 to a tip 134 at the terminal end of the distal portion 130. In some aspects, the threading 132 may be configured to be oversized relative to the circumference/taper of the tapered cylindrical geometry of the distal portion 130. The tip 134 may include a tap 136 (e.g., a self-tapping portion, cutting flute or flutes, etc.) configured to facilitate implantation within an intramedullary canal of a bone (e.g., so as to eliminate the need for a pilot hole or facilitate implantation of the distal portion 130 of the implant after an opening in an intramedullary canal is punched rather than drilled).

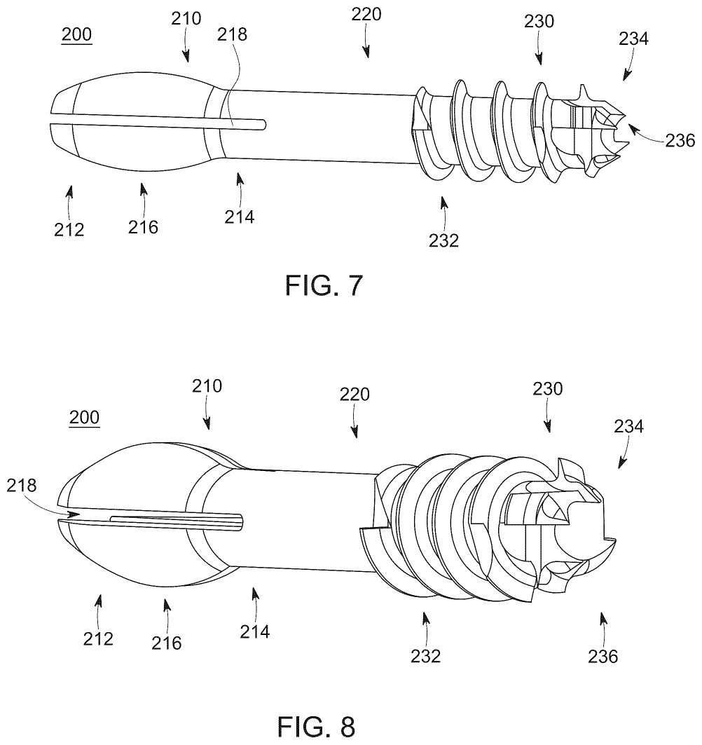

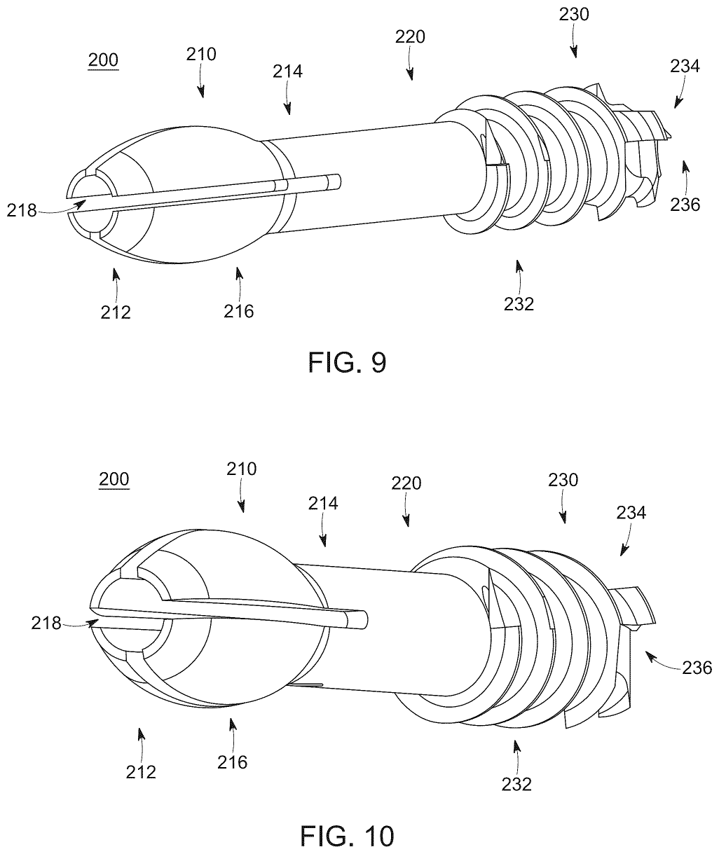

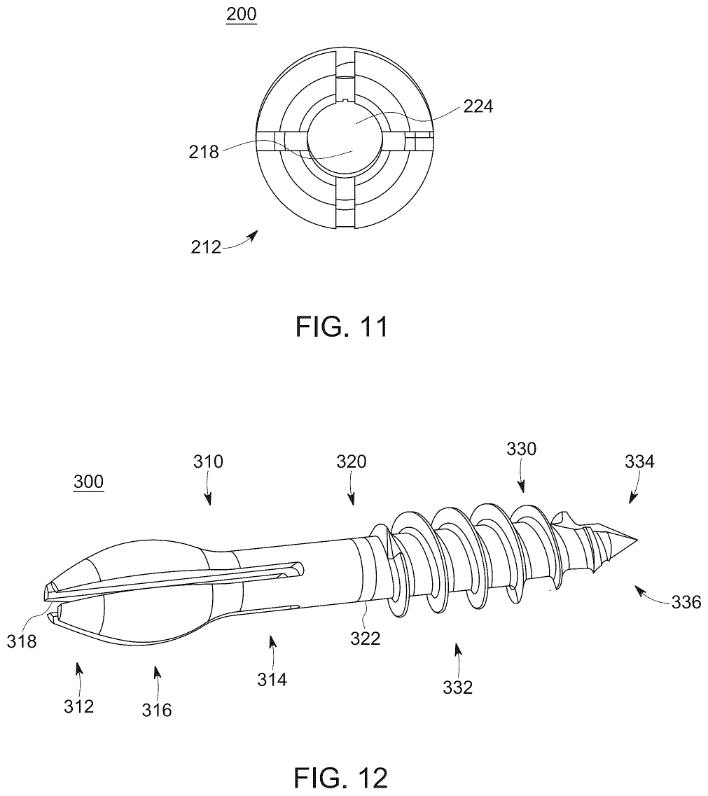

Referring now to FIGS. 6-11, an exemplary implant 200 is shown. It should be understood that, as shown and described subsequently herein, the implant 200 may be desirable in various sizes/geometries and/or other configurations in order to address various conditions, both chronic and acute, of the extremities (e.g., feet as shown in an exemplary fashion herein) as well as other portions of the anatomy. In some aspects, the implant 200 may have greater or lesser lengthwise dimensions and/or cross-sectional dimensions/geometries. For example, a portion of the implant 200 may be longer, shorter, or have greater or lesser cross-sectional dimensions than those of the implant 200 as shown with reference to FIGS. 6-11. Similarly, the implant 200 may also omit features shown and described herein and/or may include additional features.

The implant 200 is shown to include a proximal portion 210, a distal portion 230, and a central portion 220 disposed between the proximal portion 210 and the distal portion 230. The notation of proximal and distal portions 210 and 230, respectively, is arbitrary as shown in FIGS. 1-5. However, when implanted in various portions of the human anatomy, either the proximal portion 210 or the distal portion 230 may be positioned proximally (and with the other portion positioned distally) relative to the anatomy. As shown, the proximal, central, and distal portions 210, 220, and 230 are substantially centered about a common longitudinal axis. In some aspects, one or more of the proximal, central, and distal portions 210, 220, 230 may include the same and/or similar geometric shapes and/or cross-sections, for example a substantially cylindrical cross-section. As shown, each of the proximal, central, and distal portions 210, 220, and 230 are integral with one another. However, in some aspects one or more of the proximal, central, and distal portions 210, 220, and 230 may be releasably couplable with one another and/or with other components of an implant system.

The proximal portion 210 of the implant 200 is shown to extend from the central portion 220 in a direction substantially opposite from the distal portion 230. The proximal portion 210 is shown to include a plurality of projections 212 (e.g., prongs, tines, etc.) extending from the central portion 220 such that each of the plurality of projections 212 extends along an axis that is substantially parallel to the adjacent prongs in addition to being substantially parallel to the longitudinal axis of the implant 200. As shown in FIGS. 6-11, the plurality of projections 212 includes four projections, however alternate embodiments may include a lesser or greater number of projections 212. As shown, each of the plurality of projections 212 has substantially the same geometry of the other projections of the implant 200. However, in some aspects one or more projection of the plurality of projections 212 may have a different size, length, or other geometric difference from the remaining projections of the implant 200.

As shown, each of the projections 212 occupies approximately 90-degress or less of a cylinder about the longitudinal axis of the implant 200. That is to say that each of the projections 212 is positioned in a separate quadrant of such a cylinder or a circular cross-section thereof. However, as mentioned, an alternate number of projections 212 may result in alternate geometric configurations (e.g., two projections with each occupying approximately 180-degrees or less). The plurality of projections 212 are spaced from one another by a cavity 218 which, as shown, occupies the space of the longitudinal axis of the implant 200 as it extends through the proximal portion 210. The cavity 188 may include a substantially cross-shaped (e.g., a plus-sign) geometry with each projection of the cross extending outwardly from the longitudinal axis of the implant 200 and between two projections of the plurality of projections. Accordingly, the cavity 218 may have alternate geometries should the implant 200 include an alternate number of projections. The implant 200 is further shown to include a cannulation 224 extending along the longitudinal axis of the implant 200 for the length of the implant (e.g., in fluid communication with the cavity 218 and establishing fluid communication along and/or around the longitudinal axis between opposing ends of the proximal and distal portions 210 and 230, respectively). In some aspects, the cannulation 224 may be configured to accommodate a portion of an instrument (e.g., to releasably couple with, etc.) in order to facilitate implantation of the implant 200.

Each projection of the plurality of projections 212 is shown to include a first portion 214 (e.g., a linear portion) and a second portion 216 (e.g., a rounded portion). Each projection of the plurality of projections 212 as shown includes substantially the same geometry and, for the sake of brevity, a single projection of the plurality of projections 212 will be described in detail subsequently. The linear portion 214 is shown to be integral with the central portion 220 of the implant 200 and, as mentioned previously, extend from the central portion 220 in a direction substantially opposite that of the distal portion 230 as well as substantially parallel to the longitudinal axis of the implant 200. As shown, the linear portion 214 is substantially shorter in the longitudinal direction than the liner portion 14 of the implant 100. However, both the linear portions 114 and 214 may include various lengths. The linear portion 214 is shown to include three main surfaces, with two forming a substantially orthogonal angle with one another adjacent the longitudinal axis of the implant 100. The third surface is shown to be a substantially rounded, convex surface (which may correspond to a radius of the central portion 220 or other portions of the implant 200) that extends between the edges of the two orthogonal surfaces. In some aspects, each linear portion may have a geometry the same as and/or similar to a quartered section of a cylinder, and may also include a corresponding cross-sectional geometry.

The rounded portion 216 of each projection of the plurality of projections will, for the sake of brevity, also be described with reference to a single projection. The rounded portion 216 is shown to have a substantially greater lateral dimension than that of the linear portion 214 (e.g., extends further radially from the longitudinal axis of the implant 200). The outer surface of the rounded portion has a geometry similar to a fraction of the outer surface of a bulbous or elliptical (or ellipsoid) shape. With respect to the four rounded portions 216 as a whole, each includes a substantially equal surface area on the rounded portion 216 which, collectively, make up most of the outer surface of the bulbous/elliptical shape (with the portions of the cavity 218 occupying the voids). The rounded portion 216 as shown is substantially symmetrical about a line of symmetry configured perpendicular to the longitudinal axis of the implant, where said line of symmetry corresponds to a point at which the circumference/radius of the bulbous/ellipsoid shape is greatest (and said circumference/radius decreases when moving along the longitudinal axis of the implant 200 in either direction from the line of symmetry).

The plurality of projections 212 are configured such that one or more of the projections may be depressed inward (e.g., toward the longitudinal axis of the implant) so as to decrease the lateral dimension of the proximal portion 210 of the implant 200. For example, a pilot hole may be created in a portion of a bone (e.g., an intramedullary canal) with a lateral dimension less than that of the proximal portion 210 at its greatest point (the rounded portion 216) when in an un-depressed state. Accordingly, the implant 200 may be implanted in such an opening/pilot hole by depressing one or more of the plurality projections 212 (specifically, the rounded portion 216) so as to decrease the lateral dimension of the proximal portion 210 thus facilitating implantation of the proximal portion 210 within the opening/pilot hole. Once within the opening/pilot hole, the plurality of projections 212 may exhibit a resilient property, returning to the same lateral dimension as prior to depression. In some aspects, the plurality of projections 212 may return to a semi-depressed state after implantation, with a force directed radially outward from the longitudinal axis of the implant facilitating implantation and retention of the proximal portion 210 of the implant 200 within the opening/pilot hole (thus preventing pull-out/increasing pull-out resistance/strength).

The central portion 220 of the implant 200 is shown to have a substantially cylindrical geometry extending from an end of the linear portion 214 of the proximal portion 210 to a proximal-most portion of the distal portion 230 of the implant 200. As shown, the central portion 220 has a longitudinal dimension approximately equal to that that of the proximal and distal portions 210, 230. However, in alternate embodiments, the central portion 220 may include a greater or lesser longitudinal dimension than shown in FIGS. 6-11. Conversely, in some embodiments the central portion 220 may include a lesser longitudinal dimension than shown or, in some aspects, may be absent from the implant 200 (e.g., the proximal and distal portions 210, 230 are integral with one another. In some aspects, the implant 200 may include a marking on the central portion 220 the same as and/or similar to that of the implant 100, with said marking arranged on an outer surface thereof. The marking may span the circumference of the central portion 220 and may function as a depth indicator to aid in insertion of the implant 200. In some aspects, the marking may include multiple markings, for example multiple rings indicating various depths of insertion when the implant 200 is implanted.

The distal portion 230 is shown to include a substantially cylindrical geometry having a lateral dimension the same as or similar to that of the central portion 220. In some aspects, the distal portion may include a substantially tapered geometry, with said geometry tapering toward the end of the distal portion 230 opposite the central portion 220. The distal portion is further shown to include a threading 232 extending circumferentially (e.g., as a spiral configuration) from a portion adjacent the central portion 220 to a tip 234 at the terminal end of the distal portion 230. In some aspects, the threading 232 may be configured to be oversized relative to the circumference/taper of the tapered cylindrical geometry of the distal portion 230. The tip 234 may include a tap 236 (e.g., a self-tapping portion, cutting flute or flutes, etc.) configured to facilitate implantation within an intramedullary canal of a bone (e.g., so as to eliminate the need for a pilot hole or facilitate implantation of the distal portion 230 of the implant after an opening in an intramedullary canal is punched rather than drilled).

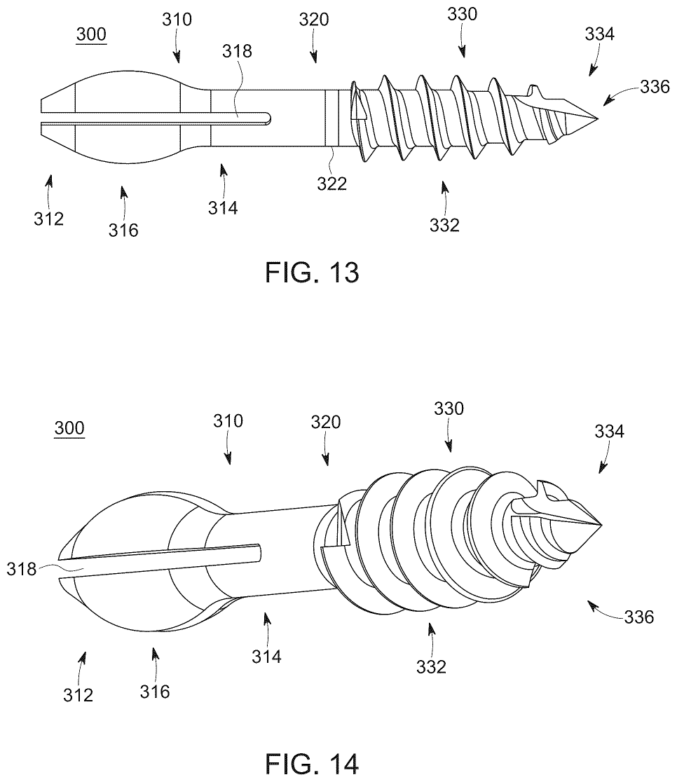

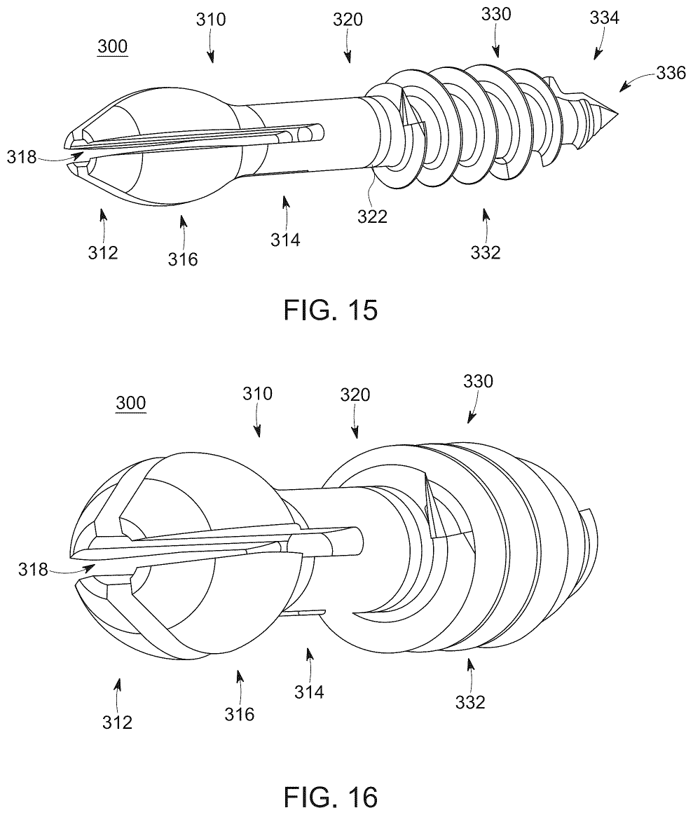

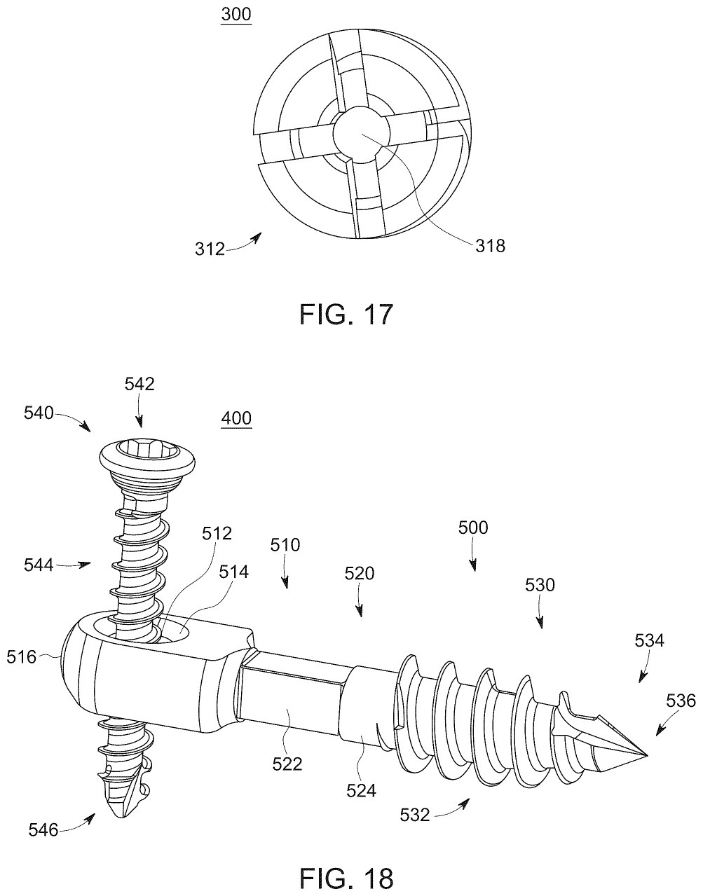

Referring now to FIGS. 12-17, an exemplary implant 300 is shown. It should be understood that, as shown and described subsequently herein, the implant 300 may be desirable in various sizes/geometries and/or other configurations in order to address various conditions, both chronic and acute, of the extremities (e.g., feet as shown in an exemplary fashion herein) as well as other portions of the anatomy. In some aspects, the implant 300 may have greater or lesser lengthwise dimensions and/or cross-sectional dimensions/geometries. For example, a portion of the implant 300 may be longer, shorter, or have greater or lesser cross-sectional dimensions than those of the implant 300 as shown with reference to FIGS. 12-17. Similarly, the implant 300 may also omit features shown and described herein and/or may include additional features.

The implant 300 is shown to include a proximal portion 310, a distal portion 330, and a central portion 320 disposed between the proximal portion 310 and the distal portion 330. The notation of proximal and distal portions 310 and 330, respectively, is arbitrary as shown in FIGS. 12-17. However, when implanted in various portions of the human anatomy, either the proximal portion 310 or the distal portion 330 may be positioned proximally (and with the other portion positioned distally) relative to the anatomy. As shown, the proximal, central, and distal portions 310, 320, and 330 are substantially centered about a common longitudinal axis. In some aspects, one or more of the proximal, central, and distal portions 310, 320, 330 may include the same and/or similar geometric shapes and/or cross-sections, for example a substantially cylindrical cross-section. As shown, each of the proximal, central, and distal portions 310, 320, and 330 are integral with one another. However, in some aspects one or more of the proximal, central, and distal portions 310, 320, and 330 may be releasably couplable with one another and/or with other components of an implant system.

The proximal portion 310 of the implant 300 is shown to extend from the central portion 320 in a direction substantially opposite from the distal portion 330. The proximal portion 310 is shown to include a plurality of projections 312 (e.g., prongs, tines, etc.) extending from the central portion 320 such that each of the plurality of projections 312 extends along an axis that is substantially parallel to the adjacent prongs in addition to being substantially parallel to the longitudinal axis of the implant 300. As shown in FIGS. 1-5, the plurality of projections 312 includes four projections, however alternate embodiments may include a lesser or greater number of projections 312. As shown, each of the plurality of projections 312 has substantially the same geometry of the other projections of the implant 300. However, in some aspects one or more projection of the plurality of projections 312 may have a different size, length, or other geometric difference from the remaining projections of the implant 300.

As shown, each of the projections 312 occupies approximately 90-degress or less of a cylinder about the longitudinal axis of the implant 300. That is to say that each of the projections 312 is positioned in a separate quadrant of such a cylinder or a circular cross-section thereof. However, as mentioned, an alternate number of projections 312 may result in alternate geometric configurations (e.g., two projections with each occupying approximately 180-degrees or less). The plurality of projections 312 are spaced from one another by a cavity 318 which, as shown, occupies the space of the longitudinal axis of the implant 300 as it extends through the proximal portion 310. The cavity 188 may include a substantially cross-shaped (e.g., a plus-sign) geometry with each projection of the cross extending outwardly from the longitudinal axis of the implant 300 and between two projections of the plurality of projections. Accordingly, the cavity 318 may have alternate geometries should the implant 300 include an alternate number of projections.

Each projection of the plurality of projections 312 is shown to include a first portion 314 (e.g., a linear portion) and a second portion 316 (e.g., a rounded portion). Each projection of the plurality of projections 312 as shown includes substantially the same geometry and, for the sake of brevity, a single projection of the plurality of projections 312 will be described in detail subsequently. The linear portion 314 is shown to be integral with the central portion 320 of the implant 300 and, as mentioned previously, extend from the central portion 320 in a direction substantially opposite that of the distal portion 330 as well as substantially parallel to the longitudinal axis of the implant 300. The linear portion 314 is shown to include three main surfaces, with two forming a substantially orthogonal angle with one another adjacent the longitudinal axis of the implant 300. The third surface is shown to be a substantially rounded, convex surface (which may correspond to a radius of the central portion 320 or other portions of the implant 300) that extends between the edges of the two orthogonal surfaces. In some aspects, each linear portion may have a geometry the same as and/or similar to a quartered section of a cylinder, and may also include a corresponding cross-sectional geometry.

The rounded portion 316 of each projection of the plurality of projections will, for the sake of brevity, also be described with reference to a single projection. The rounded portion 316 is shown to have a substantially greater lateral dimension than that of the linear portion 314 (e.g., extends further radially from the longitudinal axis of the implant 300). The outer surface of the rounded portion has a geometry similar to a fraction of the outer surface of a bulbous or elliptical (or ellipsoid) shape. With respect to the four rounded portions 316 as a whole, each includes a substantially equal surface area on the rounded portion 316 which, collectively, make up most of the outer surface of the bulbous/elliptical shape (with the portions of the cavity 318 occupying the voids). The rounded portion 316 as shown is substantially symmetrical about a line of symmetry configured perpendicular to the longitudinal axis of the implant, where said line of symmetry corresponds to a point at which the circumference/radius of the bulbous/ellipsoid shape is greatest (and said circumference/radius decreases when moving along the longitudinal axis of the implant 300 in either direction from the line of symmetry).

The plurality of projections 312 are configured such that one or more of the projections may be depressed inward (e.g., toward the longitudinal axis of the implant) so as to decrease the lateral dimension of the proximal portion 310 of the implant 300. For example, a pilot hole may be created in a portion of a bone (e.g., an intramedullary canal) with a lateral dimension less than that of the proximal portion 310 at its greatest point (the rounded portion 316) when in an un-depressed state. Accordingly, the implant 300 may be implanted in such an opening/pilot hole by depressing one or more of the plurality projections 312 (specifically, the rounded portion 316) so as to decrease the lateral dimension of the proximal portion 310 thus facilitating implantation of the proximal portion 310 within the opening/pilot hole. Once within the opening/pilot hole, the plurality of projections 312 may exhibit a resilient property, returning to the same lateral dimension as prior to depression. In some aspects, the plurality of projections 312 may return to a semi-depressed state after implantation, with a force directed radially outward from the longitudinal axis of the implant facilitating implantation and retention of the proximal portion 310 of the implant 300 within the opening/pilot hole (thus preventing pull-out/increasing pull-out resistance/strength).

The central portion 320 of the implant 300 is shown to have a substantially cylindrical geometry extending from an end of the linear portion 314 of the proximal portion 310 to a proximal-most portion of the distal portion 330 of the implant 300. As shown, the central portion 320 has a lesser longitudinal dimension than that of the proximal and distal portions 310, 330. However, in alternate embodiments, the central portion 320 may include a greater longitudinal dimension than shown in FIGS. 12-17. Conversely, in some embodiments the central portion 320 may include a lesser longitudinal dimension than shown or, in some aspects, may be absent from the implant 300 (e.g., the proximal and distal portions 310, 330 are integral with one another. The central portion 320 is shown to include a marking 322 arranged on an outer surface thereof. As shown, the marking 322 spans the circumference of the central portion 320 and may function as a depth indicator to aid in insertion of the implant 300. In some aspects, the marking 322 may include multiple markings, for example multiple rings indicating various depths of insertion when the implant 300 is implanted.

The distal portion 330 is shown to include a substantially cylindrical geometry adjacent the central portion 320, with said geometry tapering toward the end of the distal portion 330 opposite the central portion 320. The distal portion is further shown to include a threading 332 extending circumferentially (e.g., as a spiral configuration) from a portion adjacent the central portion 320 to a tip 334 at the terminal end of the distal portion 330. In some aspects, the threading 332 may be configured to be oversized relative to the circumference/taper of the tapered cylindrical geometry of the distal portion 330. The tip 334 may include a tap 336 (e.g., a self-tapping portion, cutting flute or flutes, etc.) configured to facilitate implantation within an intramedullary canal of a bone (e.g., so as to eliminate the need for a pilot hole or facilitate implantation of the distal portion 330 of the implant after an opening in an intramedullary canal is punched rather than drilled).

It should be understood that the implant 300 may include one or more features the same as and/or similar to those of the implant 100 as shown and described previously herein. Similarly, the implant 300 may include the same and/or similar to features to those of the implant 300, but with one or more of said features being of a different size, geometry, or other spatial characteristic. For example, the central portion 320 of the implant 300 may have a greater or lesser diameter than that of the central portion 120 of the implant 100. Further, the central portion 320 may have a greater or lesser longitudinal dimension than that of the central portion 120. In some aspects, the implant 300 and the implant 100 may be the same implant but, as mentioned previously, with components thereof varying in size (e.g., length, width, cross-sectional area, etc.) relative to the corresponding components of the implant 100. Accordingly, the implant 300 may be of a substantially smaller size (e.g., all components proportionally smaller than the implant 100) in order to accommodate for smaller portions of the anatomy (where similarly, the implant 100 and components thereof may be larger proportionally in order to accommodate larger portions of the anatomy).

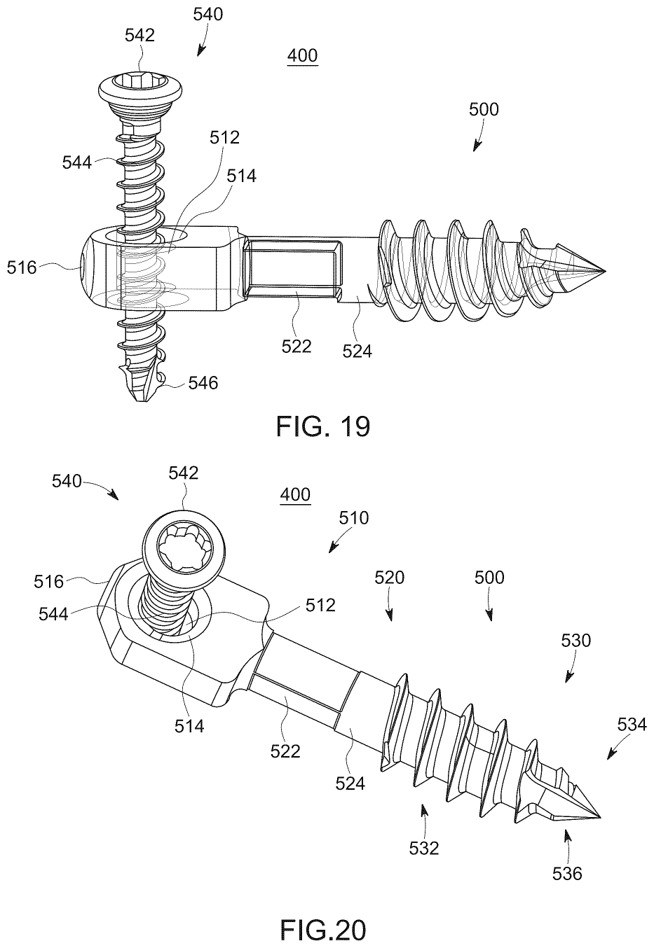

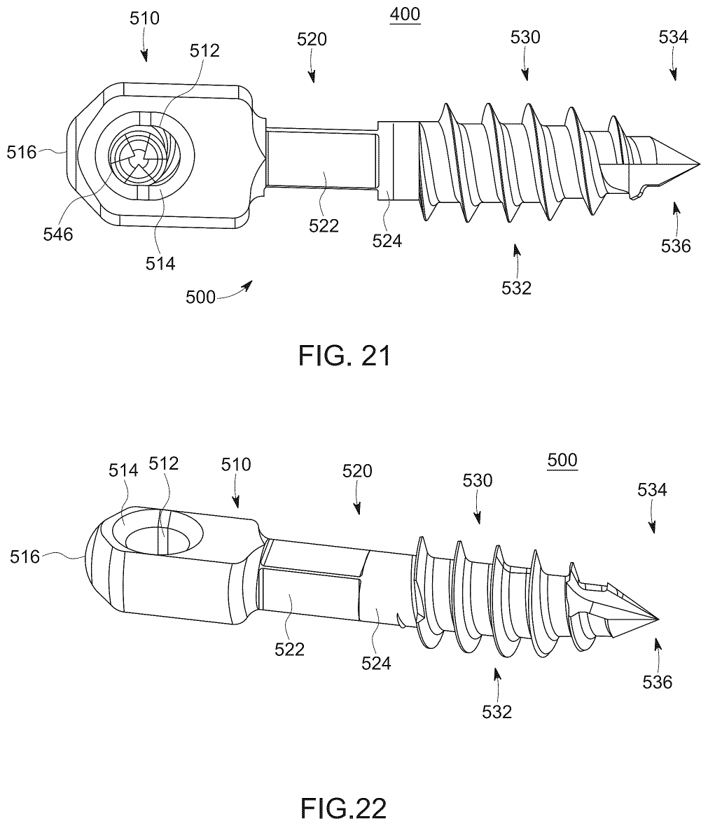

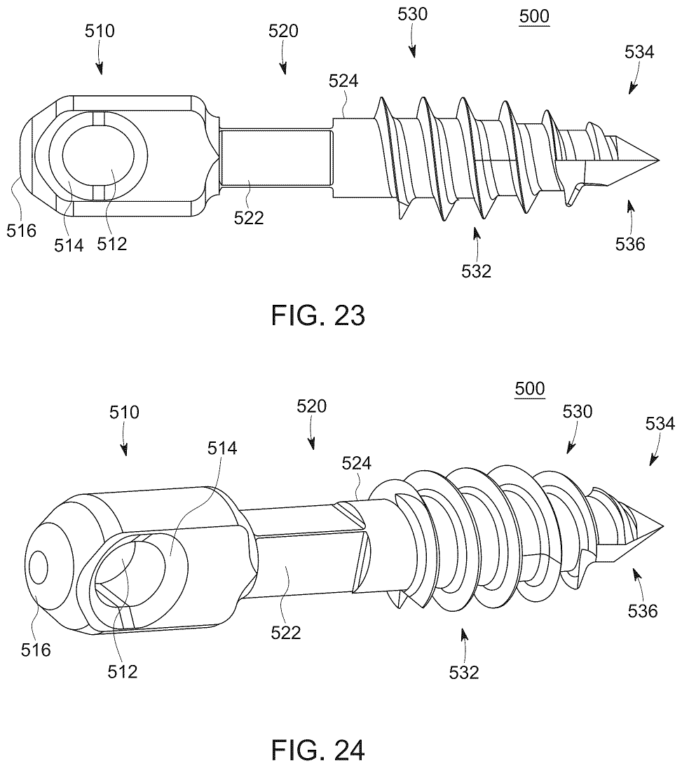

Referring now to FIGS. 18-24, an implant system 400 is shown, according to an exemplary embodiment. The implant system 400 is shown to include an implant 500 as well as a screw (e.g., a fastener, etc.) 540. As shown, the screw 540 includes a head 542 as well as a threading 544 extending along a length of the screw and terminating at a tip 546, with the tip 546 positioned substantially opposite the threading 544 from the head 542. As shown in FIGS. 18-21, at least a portion of the screw 540 (e.g., the threading 544 and the portion of the screw 540 on which the threading 540 is disposed on the outer surface thereof) is configured to releasably couple the implant 500 with the screw 540. Further, the screw 540 may be configured to couple with the implant 500 via one or more bones of portions of bone of a patient so as to provide compression of two bony components toward one another. For example, a first portion of the implant 500 may be coupled with a first bony portion, and a second portion of the implant 500 may be coupled with a second bony portion via the screw 540 (which is thus coupled with both the implant and the second bony portion) so as to provide compression between the first and second bony portions via the implant 500 and the screw 540.

The implant 500 is shown to include a proximal portion 510, a distal portion 530, and a central portion 520 disposed between the proximal portion 510 and the distal portion 530. The notation of proximal and distal portions 510 and 530, respectively, is arbitrary as shown in FIGS. 18-24. However, when implanted in various portions of the human anatomy, either the proximal portion 510 or the distal portion 530 may be positioned proximally (and with the other portion positioned distally) relative to the anatomy. As shown, the proximal, central, and distal portions 510, 520, and 530 are substantially centered about a common longitudinal axis. In some aspects, one or more of the proximal, central, and distal portions 510, 520, 530 may include the same and/or similar geometric shapes and/or cross-sections, for example a substantially cylindrical cross-section. As shown, each of the proximal, central, and distal portions 510, 520, and 530 are integral with one another. However, in some aspects one or more of the proximal, central, and distal portions 510, 520, and 530 may be releasably couplable with one another and/or with other components of an implant system.

The proximal portion 510 of the implant 500 is shown to extend from the central portion 520 in a direction substantially opposite from the distal portion 530. As shown, the proximal portion 510 includes a substantially greater lateral dimension than that of the central portion 520 in at least one direction (e.g., wider in at least a medial, lateral, anterior and/or posterior direction with said directions assigned arbitrarily; or as shown “wider” in left and right/horizontal directions and/or “taller” in top/bottom directions). The proximal portion is shown to include an opening 512 shown as a through hole centrally positioned in the proximal portion 510 and extending from one surface (e.g., a top surface) through the proximal portion 510 to a second opposite surface (e.g., a bottom surface). As shown, the opening 512 is positioned such that a longitudinal axis of the opening 512 (e.g., an axis about which the opening 512—which is shown as a circular/cylindrical opening, but may include other geometries in alternate embodiments—is centered concentrically about) would be positioned substantially perpendicular (e.g., in perpendicular planes) relative to the longitudinal axis of the implant 500. In some aspects, the longitudinal axis of the opening 512 may intersect with the longitudinal axis of the implant 500. The opening 512 is configured to receive at least a portion of a fastener, for example the screw 540, therethrough such that at least a portion of the screw 540 abuts a portion of the proximal portion 510 that defines a lateral dimension of the opening 512 (e.g., contacts the threading/shaft portion of the screw 540 as shown).

The proximal portion 510 is further shown to include a tapered edge 514 configured adjacent the opening 512. As shown, the entrance to the opening 512 relative to a top or bottom surface (e.g., a surface in a plane perpendicular to the longitudinal axis of the opening 512) of the proximal portion 510 is shown to have a tapered edge 514 (e.g., a graduated, rounded, or otherwise non-perpendicular interface between the top/bottom surfaces of the proximal portion 512 and the surfaces of the proximal portion 510 that laterally define the opening 512. The proximal portion 510 is also shown to include a tip 516 arranged at an end of the proximal portion 510 opposite that of the interface with the central portion 520. The tip 516 is shown to have a tapered geometry where the lateral dimension of the proximal portion 510 at the top 516 is lesser than that of the proximal portion directly adjacent the opening 512 or the interface with the central portion 520.

The central portion 520 of the implant 500 is shown to have a substantially rectanguloid geometry extending from an end of the proximal portion 510 to a proximal-most portion of the distal portion 530 of the implant 500. As shown, the central portion 520 has a lesser longitudinal dimension than that of the proximal 510. However, in alternate embodiments, the central portion 520 may include a greater longitudinal dimension than shown in FIGS. 18-24. Conversely, in some embodiments the central portion 520 may include a lesser longitudinal dimension than shown or, in some aspects, may be absent from the implant 500 (e.g., the proximal and distal portions 510, 530 are integral with one another. The central portion 520 is shown to include a plurality of flats 522 (shown as four flats) arranged about the central portion 520 (e.g., outer surfaces of the rectanguloid). In some aspects, the flats 522 may be configured to interface with or facilitate releasable coupling with one or more instruments incorporated in conjunction with the system 400. Further, in some embodiments the central portion 520 may include alternate geometries to the rectanguloid shown, in which case the central portion 520 may include a lesser or greater number of flats 522 than shown (or such an embodiment may also include four flats as shown).

The central portion 520 is further shown to include a cylindrical portion 524 arranged between the plurality of flats 522 and the distal portion 530. In some aspects, the plurality of flats 522 may include a tapered transition into the cylindrical portion 524. The central portion 520 may also include a marking arranged on an outer surface thereof. Such a marking may span the outer dimension of the central portion 520 and may function as a depth indicator to aid in insertion of the implant 500. In some aspects, such a marking may include multiple markings, for example multiple rings indicating various depths of insertion when the implant 500 is implanted.

The distal portion 530 is shown to include a substantially cylindrical geometry adjacent the central portion 520, with said geometry tapering toward the end of the distal portion 530 opposite the central portion 520. The distal portion is further shown to include a threading 532 extending circumferentially (e.g., as a spiral configuration) from a portion adjacent the central portion 520 to a tip 534 at the terminal end of the distal portion 530. In some aspects, the threading 532 may be configured to be oversized relative to the circumference/taper of the tapered cylindrical geometry of the distal portion 530. The tip 534 may include a tap 536 (e.g., a self-tapping portion, cutting flute or flutes, etc.) configured to facilitate implantation within an intramedullary canal of a bone (e.g., so as to eliminate the need for a pilot hole or facilitate implantation of the distal portion 530 of the implant after an opening in an intramedullary canal is punched rather than drilled).

The system 400 is configured such that the distal portion 530 may be rotatably coupled (via the threading 532) with a first bony fragment (e.g., an intramedullary canal which may be manipulated or “punched” to create a volume sufficient to receive and couple with the distal portion 530) such as a distal portion of a metatarsal. The proximal portion 510 is similarly configured to couple with a second bony fragment (e.g., an intramedullary canal which may be manipulated or “punched” to create a volume sufficient to receive and couple with the proximal portion 510) where the proximal portion is positioned such that an longitudinal axis of the opening 512 extends substantially perpendicular to a longitudinal axis of an anatomical opening (e.g., an intramedullary canal). The proximal portion 510 may be disposed in such an anatomical opening such that the screw 542 may be inserted along the longitudinal axis of the opening 512 (e.g., in a plane perpendicular to the longitudinal axis of the implant 500) such that a portion of the threading 544 of the screw 540 contacts a proximal most edge of the portion of the proximal portion 510 that defines the proximal-most portion of the opening 512. Accordingly, such positioning of the screw 540 biases the implant 500 in a proximal direction (relative to the implant 500) thus compressing the first and second bony fragments.

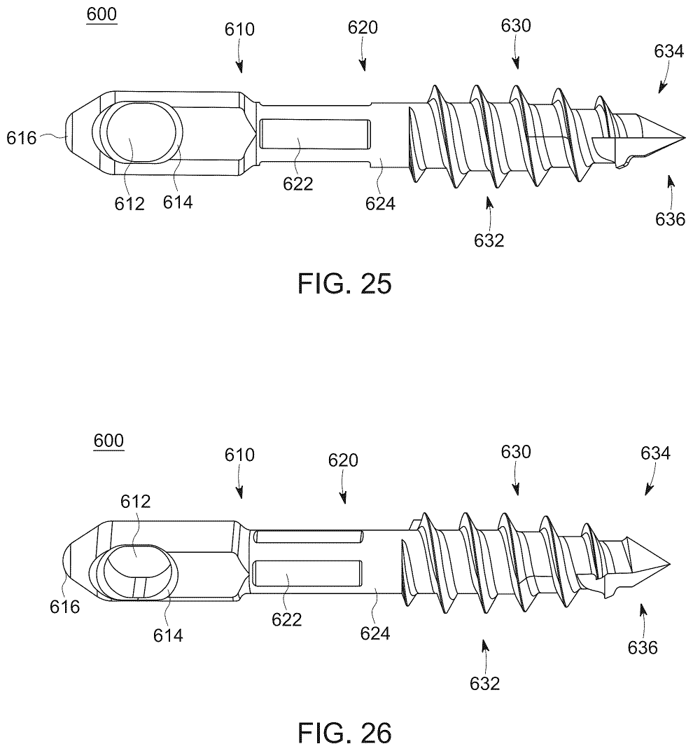

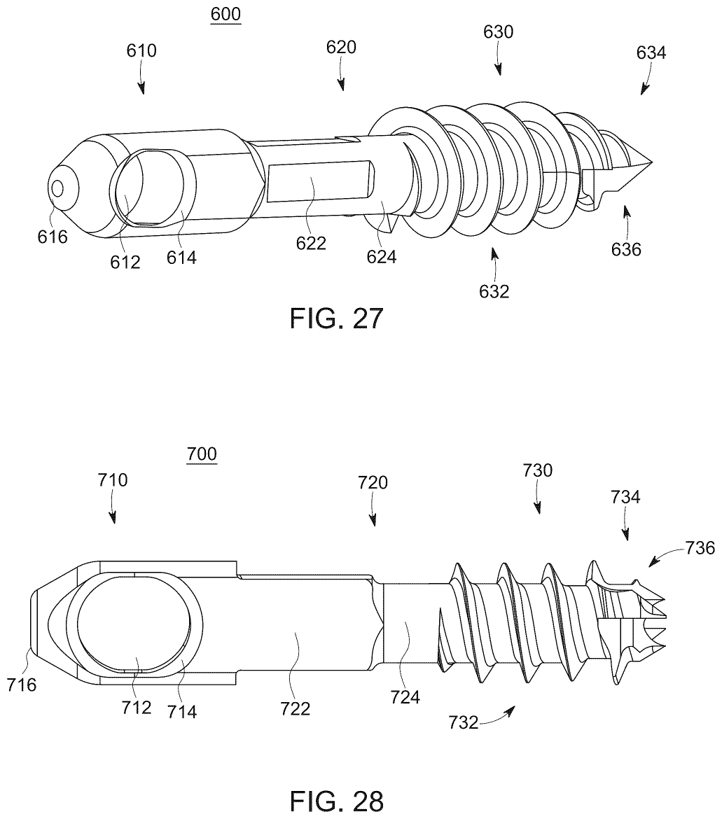

Referring now to FIGS. 25-27, an implant 600 is shown, according to an exemplary embodiment. The implant 600 may include one or more features the same as and/or similar to the implant 500 and, further, may be compatible with the screw 540 (or other fasteners) as shown and described with reference to FIGS. 18-21 and described previously herein. The implant 600 is shown to include a proximal portion 610, a distal portion 630, and a central portion 620 disposed between the proximal portion 610 and the distal portion 630. The notation of proximal and distal portions 610 and 630, respectively, is arbitrary as shown in FIGS. 25-27. However, when implanted in various portions of the human anatomy, either the proximal portion 610 or the distal portion 630 may be positioned proximally (and with the other portion positioned distally) relative to the anatomy. As shown, the proximal, central, and distal portions 610, 620, and 630 are substantially centered about a common longitudinal axis. In some aspects, one or more of the proximal, central, and distal portions 610, 620, 630 may include the same and/or similar geometric shapes and/or cross-sections, for example a substantially cylindrical cross-section. As shown, each of the proximal, central, and distal portions 610, 620, and 630 are integral with one another. However, in some aspects one or more of the proximal, central, and distal portions 610, 620, and 630 may be releasably couplable with one another and/or with other components of an implant system.

The proximal portion 610 of the implant 600 is shown to extend from the central portion 620 in a direction substantially opposite from the distal portion 630. As shown, the proximal portion 610 includes a substantially greater lateral dimension than that of the central portion 620 in at least one direction (e.g., wider in at least a medial, lateral, anterior and/or posterior direction with said directions assigned arbitrarily; or as shown “wider” in left and right/horizontal directions and/or “taller” in top/bottom directions). The proximal portion is shown to include an opening 612 shown as a through hole centrally positioned in the proximal portion 610 and extending from one surface (e.g., a top surface) through the proximal portion 610 to a second opposite surface (e.g., a bottom surface). As shown, the opening 612 is positioned such that a longitudinal axis of the opening 612 (e.g., an axis about which the opening 612—which is shown as a circular/cylindrical opening, but may include other geometries in alternate embodiments—is centered concentrically about) would be positioned substantially perpendicular (e.g., in perpendicular planes) relative to the longitudinal axis of the implant 600. In some aspects, the longitudinal axis of the opening 612 may intersect with the longitudinal axis of the implant 600. The opening 612 is configured to receive at least a portion of a fastener, for example the screw 540, therethrough such that at least a portion of the screw 540 abuts a portion of the proximal portion 610 that defines a lateral dimension of the opening 612 (e.g., contacts the threading/shaft portion of the screw 540 as shown).

The proximal portion 610 is further shown to include a tapered edge 614 configured adjacent the opening 612. As shown, the entrance to the opening 612 relative to a top or bottom surface (e.g., a surface in a plane perpendicular to the longitudinal axis of the opening 612) of the proximal portion 610 is shown to have a tapered edge 614 (e.g., a graduated, rounded, or otherwise non-perpendicular interface between the top/bottom surfaces of the proximal portion 612 and the surfaces of the proximal portion 610 that laterally define the opening 612. The proximal portion 610 is also shown to include a tip 616 arranged at an end of the proximal portion 610 opposite that of the interface with the central portion 620. The tip 616 is shown to have a tapered geometry where the lateral dimension of the proximal portion 610 at the top 616 is lesser than that of the proximal portion directly adjacent the opening 612 or the interface with the central portion 620.

The central portion 620 of the implant 600 is shown to have a substantially cylindrical geometry extending from an end of the proximal portion 610 to a proximal-most portion of the distal portion 630 of the implant 600. As shown, the central portion 620 has a lesser longitudinal dimension than that of the proximal portion 610. However, in alternate embodiments, the central portion 620 may include a greater longitudinal dimension than shown in FIGS. 25-27. Conversely, in some embodiments the central portion 620 may include a lesser longitudinal dimension than shown or, in some aspects, may be absent from the implant 600 (e.g., the proximal and distal portions 610, 630 are integral with one another. The central portion 620 is shown to include a plurality of flats 622 (shown as four flats spaced approximately 90-degrees circumferentially from a mid-point of each flat) arranged about the central portion 620 (e.g., circumference). As shown in FIGS. 25-27, each flat of the plurality of flats 622 is cut into the cylindrical geometry of the central portion 620. In some aspects, the flats 622 may be configured to interface with or facilitate releasable coupling with one or more instruments incorporated in conjunction with, for example the system 400. Further, in some embodiments the central portion 620 may include alternate geometries to the cylindrical geometry shown, in which case the central portion 620 may include a lesser or greater number of flats 622 than shown (or such an embodiment may also include four flats as shown).

The central portion 620 is further shown to include a cylindrical portion 624 arranged between the plurality of flats 622 and the distal portion 630. In some aspects, the plurality of flats 622 may include a tapered transition into the cylindrical portion 624. The central portion 620 may also include a marking arranged on an outer surface thereof. Such a marking may span the outer dimension of the central portion 620 and may function as a depth indicator to aid in insertion of the implant 600. In some aspects, such a marking may include multiple markings, for example multiple rings indicating various depths of insertion when the implant 600 is implanted.

The distal portion 630 is shown to include a substantially cylindrical geometry adjacent the central portion 620, with said geometry tapering toward the end of the distal portion 630 opposite the central portion 620. The distal portion is further shown to include a threading 632 extending circumferentially (e.g., as a spiral configuration) from a portion adjacent the central portion 620 to a tip 634 at the terminal end of the distal portion 630. In some aspects, the threading 632 may be configured to be oversized relative to the circumference/taper of the tapered cylindrical geometry of the distal portion 630. The tip 634 may include a tap 636 (e.g., a self-tapping portion, cutting flute or flutes, etc.) configured to facilitate implantation within an intramedullary canal of a bone (e.g., so as to eliminate the need for a pilot hole or facilitate implantation of the distal portion 630 of the implant after an opening in an intramedullary canal is punched rather than drilled).

The implant 600, which may be implemented similarly to the implant 500 shown with reference to the system 400, is configured such that the distal portion 630 may be rotatably coupled (via the threading 532) with a first bony fragment (e.g., an intramedullary canal which may be manipulated or “punched” to create a volume sufficient to receive and couple with the distal portion 630) such as a distal portion of a metatarsal. The proximal portion 610 is similarly configured to couple with a second bony fragment (e.g., an intramedullary canal which may be manipulated or “punched” to create a volume sufficient to receive and couple with the proximal portion 610) where the proximal portion is positioned such that an longitudinal axis of the opening 612 extends substantially perpendicular to a longitudinal axis of an anatomical opening (e.g., an intramedullary canal). The proximal portion 610 may be disposed in such an anatomical opening such that the screw 542 may be inserted along the longitudinal axis of the opening 612 (e.g., in a plane perpendicular to the longitudinal axis of the implant 600) such that a portion of the threading 544 of the screw 540 contacts a proximal most edge of the portion of the proximal portion 610 that defines the proximal-most portion of the opening 612. Accordingly, such positioning of the screw 540 biases the implant 600 in a proximal direction (relative to the implant 600) thus compressing the first and second bony fragments.

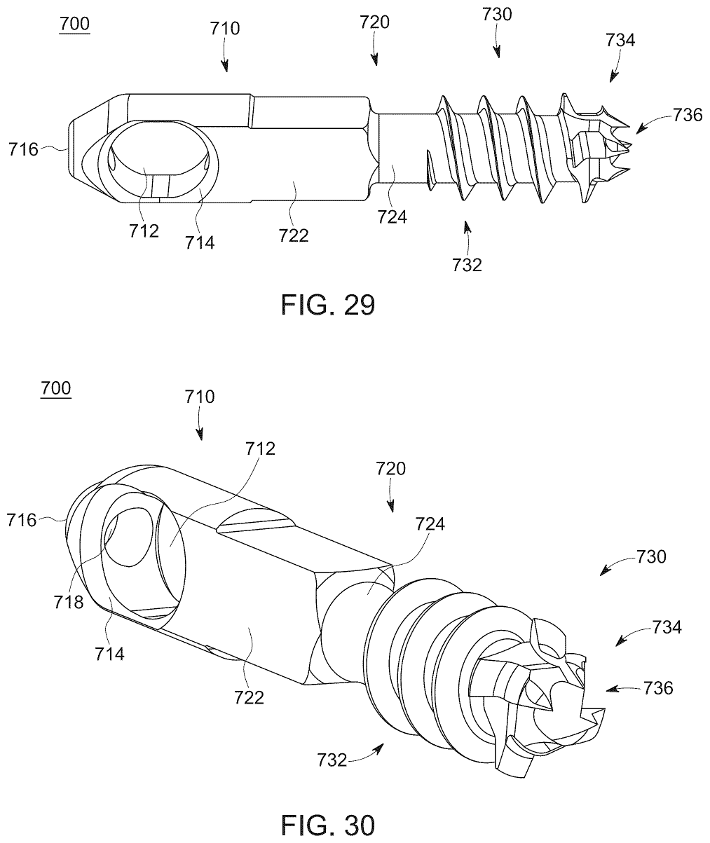

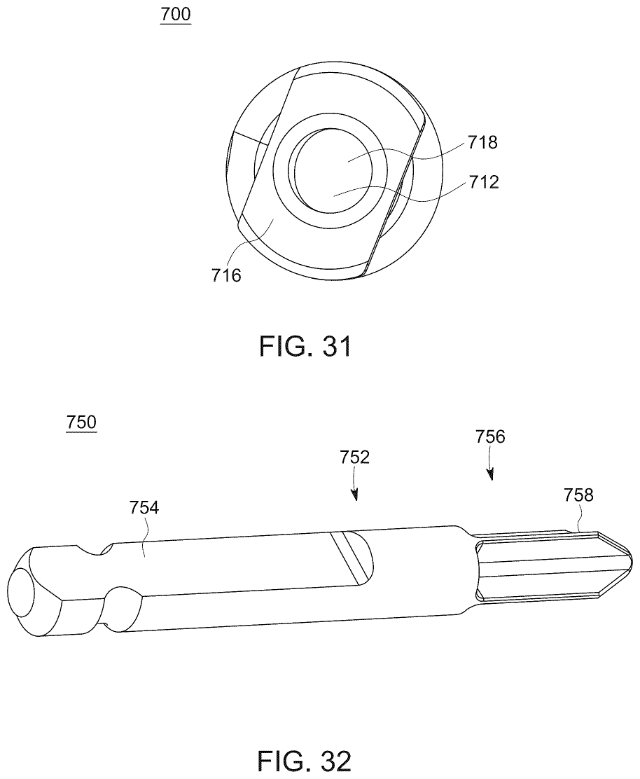

Referring now to FIGS. 28-31, an implant 700 is shown, according to an exemplary embodiment. The implant 700 may include one or more features the same as and/or similar to the implant 500 and, further, may be compatible with the screw 540 (or other fasteners) as shown and described with reference to FIGS. 18-21 and described previously herein. The implant 700 is shown to include a proximal portion 710, a distal portion 730, and a central portion 720 disposed between the proximal portion 710 and the distal portion 730. The notation of proximal and distal portions 710 and 730, respectively, is arbitrary as shown in FIGS. 28-31. However, when implanted in various portions of the human anatomy, either the proximal portion 710 or the distal portion 730 may be positioned proximally (and with the other portion positioned distally) relative to the anatomy. As shown, the proximal, central, and distal portions 710, 720, and 730 are substantially centered about a common longitudinal axis. In some aspects, one or more of the proximal, central, and distal portions 710, 720, 730 may include the same and/or similar geometric shapes and/or cross-sections, for example a substantially cylindrical cross-section. As shown, each of the proximal, central, and distal portions 710, 720, and 730 are integral with one another. However, in some aspects one or more of the proximal, central, and distal portions 710, 720, and 730 may be releasably couplable with one another and/or with other components of an implant system.

The proximal portion 710 of the implant 700 is shown to extend from the central portion 720 in a direction substantially opposite from the distal portion 730. As shown, the proximal portion 710 includes a substantially greater lateral dimension than that of the central portion 720 in at least one direction (e.g., wider in at least a medial, lateral, anterior and/or posterior direction with said directions assigned arbitrarily; or as shown “wider” in left and right/horizontal directions and/or “taller” in top/bottom directions). The proximal portion is shown to include an opening 712 shown as a through hole centrally positioned in the proximal portion 710 and extending from one surface (e.g., a top surface) through the proximal portion 710 to a second opposite surface (e.g., a bottom surface). As shown, the opening 712 is positioned such that a longitudinal axis of the opening 712 (e.g., an axis about which the opening 712—which is shown as a circular/cylindrical opening, but may include other geometries in alternate embodiments—is centered concentrically about) would be positioned substantially perpendicular (e.g., in perpendicular planes) relative to the longitudinal axis of the implant 700. In some aspects, the longitudinal axis of the opening 712 may intersect with the longitudinal axis of the implant 700. The opening 712 is configured to receive at least a portion of a fastener, for example the screw 540, therethrough such that at least a portion of the screw 540 abuts a portion of the proximal portion 710 that defines a lateral dimension of the opening 712 (e.g., contacts the threading/shaft portion of the screw 540 as shown).

The proximal portion 710 is further shown to include a tapered edge 714 configured adjacent the opening 712. As shown, the entrance to the opening 712 relative to a top or bottom surface (e.g., a surface in a plane perpendicular to the longitudinal axis of the opening 712) of the proximal portion 710 is shown to have a tapered edge 714 (e.g., a graduated, rounded, or otherwise non-perpendicular interface between the top/bottom surfaces of the proximal portion 712 and the surfaces of the proximal portion 710 that laterally define the opening 712. The proximal portion 710 is also shown to include a tip 716 arranged at an end of the proximal portion 710 opposite that of the interface with the central portion 720. The tip 716 is shown to have a tapered geometry where the lateral dimension of the proximal portion 710 at the top 716 is lesser than that of the proximal portion directly adjacent the opening 712 or the interface with the central portion 720. The tip 716 is shown to include a cannulation 718 extending along the longitudinal axis of the implant 700 from a terminal portion of the proximal portion 710 through the central portion 720 and to a terminal portion of the distal portion 730 such that the opening 712 and the cannulation are in fluid communication with one another. In some aspects, the cannulation 718 may be configured to receive a portion of an instrument, stabilization wire, or other component in order to facilitate implantation of the implant 700.

The central portion 720 of the implant 700 is shown to have a substantially rectanguloid geometry extending from an end of the proximal portion 710 to a proximal-most portion of the distal portion 730 of the implant 700. As shown, the central portion 720 has a substantially equal or slightly lesser longitudinal dimension than that of the proximal portion 710. Similarly, the central portion 720 is shown to have a substantially similar cross-sectional geometry to that of the proximal portion 710. However, in alternate embodiments, the central portion 720 may include a greater longitudinal dimension than shown in FIGS. 25-27 and/or alternate cross-sectional dimensions. Conversely, in some embodiments the central portion 720 may include a lesser longitudinal dimension than shown or, in some aspects, may be absent from the implant 700 (e.g., the proximal and distal portions 710, 730 are integral with one another. The central portion 720 is shown to include a plurality of flats 722 (shown as four flats spaced at approximately 90-degree angles from one another) arranged about the central portion 720 (e.g., outer surface). In some aspects, the flats 722 may be configured to interface with or facilitate releasable coupling with one or more instruments incorporated in conjunction with, for example the system 400. Further, in some embodiments the central portion 720 may include alternate geometries to the cylindrical geometry shown, in which case the central portion 720 may include a lesser or greater number of flats 722 than shown (or such an embodiment may also include four flats as shown).

The central portion 720 is further shown to include a cylindrical portion 724 arranged between the plurality of flats 722 and the distal portion 730. In some aspects, the plurality of flats 722 may include a tapered transition into the cylindrical portion 724. The central portion 720 may also include a marking arranged on an outer surface thereof. Such a marking may span the outer dimension of the central portion 720 and may function as a depth indicator to aid in insertion of the implant 700. In some aspects, such a marking may include multiple markings, for example multiple rings indicating various depths of insertion when the implant 700 is implanted.

The distal portion 730 is shown to include a substantially cylindrical geometry adjacent the central portion 720, with said geometry tapering toward the end of the distal portion 730 opposite the central portion 720. The distal portion is further shown to include a threading 732 extending circumferentially (e.g., as a spiral configuration) from a portion adjacent the central portion 720 to a tip 734 at the terminal end of the distal portion 730. In some aspects, the threading 732 may be configured to be oversized relative to the circumference/taper of the tapered cylindrical geometry of the distal portion 730. The tip 734 may include a tap 736 (e.g., a self-tapping portion, cutting flute or flutes, etc.) configured to facilitate implantation within an intramedullary canal of a bone (e.g., so as to eliminate the need for a pilot hole or facilitate implantation of the distal portion 730 of the implant after an opening in an intramedullary canal is punched rather than drilled).

The implant 700, which may be implemented similarly to the implant 500 shown with reference to the system 400, is configured such that the distal portion 730 may be rotatably coupled (via the threading 532) with a first bony fragment (e.g., an intramedullary canal which may be manipulated or “punched” to create a volume sufficient to receive and couple with the distal portion 730) such as a distal portion of a metatarsal. The proximal portion 710 is similarly configured to couple with a second bony fragment (e.g., an intramedullary canal which may be manipulated or “punched” to create a volume sufficient to receive and couple with the proximal portion 710) where the proximal portion is positioned such that an longitudinal axis of the opening 712 extends substantially perpendicular to a longitudinal axis of an anatomical opening (e.g., an intramedullary canal). The proximal portion 710 may be disposed in such an anatomical opening such that the screw 542 may be inserted along the longitudinal axis of the opening 712 (e.g., in a plane perpendicular to the longitudinal axis of the implant 700) such that a portion of the threading 544 of the screw 540 contacts a proximal most edge of the portion of the proximal portion 710 that defines the proximal-most portion of the opening 712. Accordingly, such positioning of the screw 540 biases the implant 700 in a proximal direction (relative to the implant 700) thus compressing the first and second bony fragments.

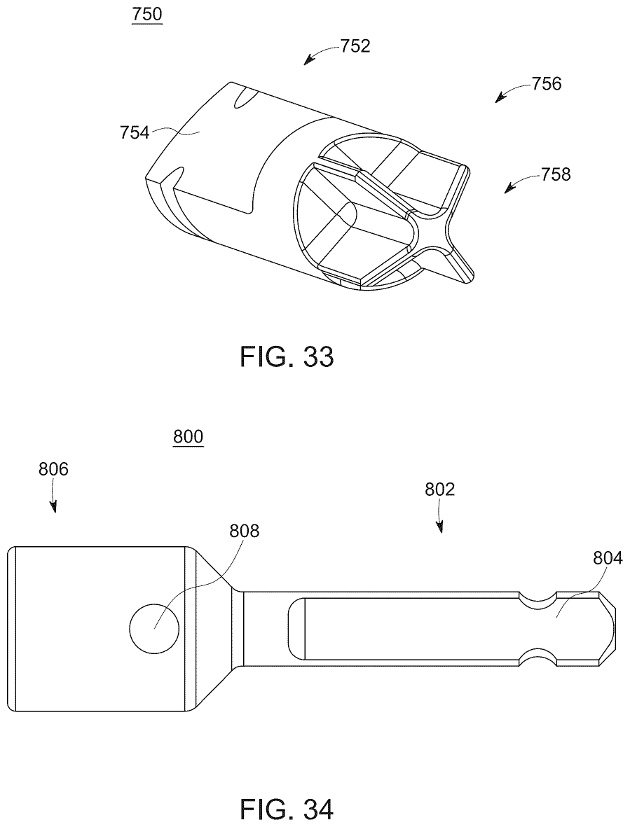

Referring now to FIGS. 32-33, an instrument 750 is shown (e.g., an inserter), according to an exemplary embodiment. The instrument 750 is shown to include a first portion 752 and a second portion 756, with both the first and second portions 752, 756 centered about a longitudinal axis of the instrument 750. The first portion 752 is shown to include a flat 754 configured to facilitate releasable coupling with a handle and/or portion of another instrument such as that shown and described subsequently herein. In some aspects, the first portion 752 may include a substantially cylindrical geometry, with the flat 754 cut into the cylindrical geometry to for a semi-spherical geometry. In some aspects, the flat 754 may include two or more flats. The second portion 756 is shown to include an engagement feature 758 which, as shown, include a plurality of fins (four as shown). In some aspects, the engagement feature 758 may be the same as and/or similar to a standard engagement feature (e.g., hexalobe, Torxx, or, as shown, Phillips head). The engagement feature 758 is configured to interface with an implant (e.g., releasably couple) such as, for example (but not limited to), the implants 100, 200, and 300 as shown and described herein. When releasably coupling with the implant 100, for example, the plurality of fins of the engagement feature 758 are configured to be received within the cavity 118 and adjacent the projections 112. Accordingly, the implant 100 may then be manipulated in a fixed position (e.g., such that the threads 132 are releasably coupled with a bony fragment).

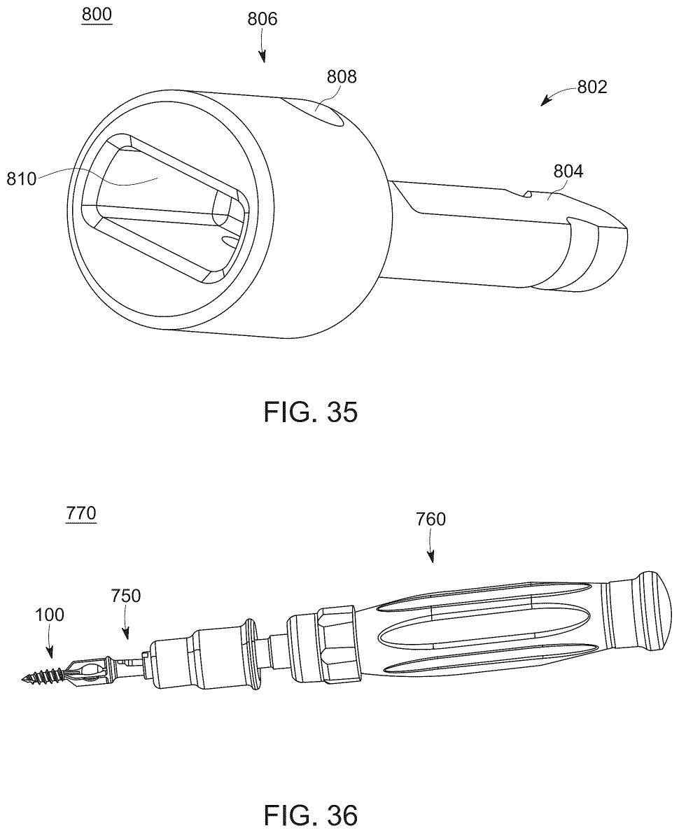

Referring now to FIGS. 34-35, an instrument 800 is shown, according to an exemplary embodiment. The instrument 800 is shown to include a first portion 802 and a second portion 806, with both the first and second portions 802, 806 centered about a longitudinal axis of the instrument 800. The first portion 802 is shown to include a flat 804 configured to facilitate releasable coupling with a handle and/or portion of another instrument such as that shown and described subsequently herein. In some aspects, the first portion 802 may include a substantially cylindrical geometry, with the flat 804 cut into the cylindrical geometry to for a semi-spherical geometry. In some aspects, the flat 804 may include two or more flats. The second portion 806 is shone to include a greater lateral dimension than that of the first portion 802. Further, the second portion 806 is shown to include a through hole 808 as well as a recess 810. The through hole 808 is shown to extend diametrically through the second portion 806 (e.g., substantially perpendicular to the longitudinal axis of the instrument 800). As shown, the recess 810 extends into the second portion 806 from the terminal end of the second portion 806 of the instrument 800. As shown, the recess 810 includes a substantially oblong or rounded rectangular cross-sectional geometry such that it may receive at least a portion of the implant 500 (or other implants including but not limited to those shown and described herein). When received in the recess 810, the longitudinal axis of the opening 512 of the implant 500 may align with the longitudinal axis of the through hole 808 such that a rigid member (e.g., the screw 540 or similar) may be inserted through the through hole 808 and the opening 512 thus retaining the implant 100 with at least a portion of the proximal portion 510 of the implant disposed within the recess 810. Accordingly, the implant 500 may then be manipulated in a fixed position (e.g., such that the threads 532 are releasably coupled with a bony fragment).

Referring now to FIG. 36, a system 770 is shown, according to an exemplary embodiment. The system 770 is shown to include a handle 760 which may include a ratcheting mechanism or other features common to surgical handles configured to facilitate rotational movement of other components. The instrument 750 is shown to be releasably coupled with the handle 760 via the first portion 752 (and, more specifically, the flat 754) where at least a portion of the first portion 752 is received and releasably retained within a portion of the handle 760. The instrument 750 is shown to be releasably coupled with the implant 100 via the engagement feature 758 (and the plurality of fins thereof) releasably coupling with the projections 112/cavity 118 of the proximal portion of the implant 100. Accordingly, the distal portion 130 of the implant 100 may be releasably coupled with a portion of bone (e.g., screwed in using the handle 760) with the implant then released from the releasable coupling with the engagement feature 758 of the instrument 750.

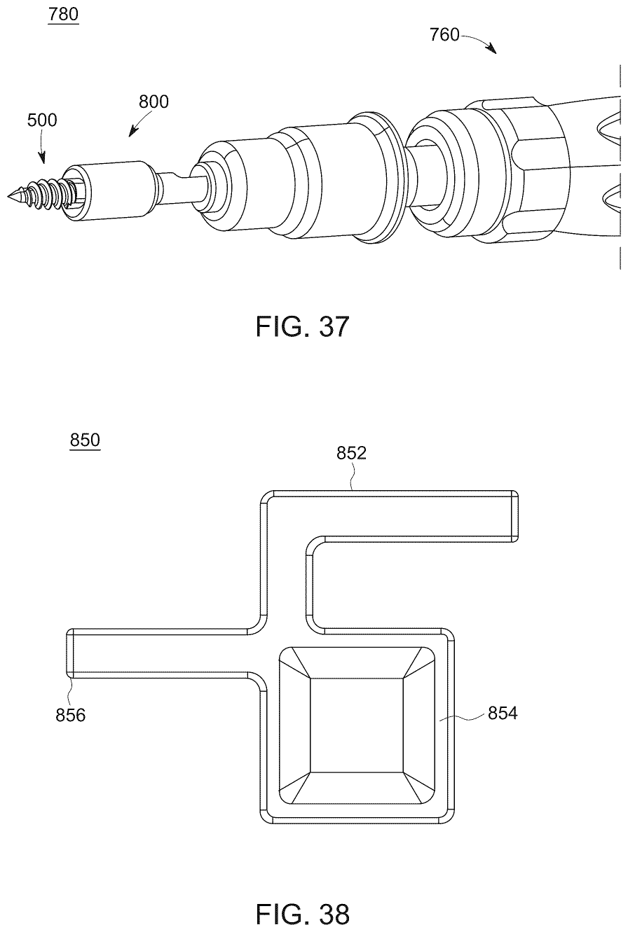

Referring now to FIG. 37, a system 780 is shown, according to an exemplary embodiment. The system 780 is shown to include the handle 760 which, as mentioned previously, may include a ratcheting mechanism or other features common to surgical handles configured to facilitate rotational movement of other components. The instrument 800 is shown to be releasably coupled with the handle 760 via the first portion 802 (and, more specifically, the flat 804) where at least a portion of the first portion 802 is received and releasably retained within a portion of the handle 760. The instrument 800 is shown to be releasably coupled with the implant 500 via the recess 810 receiving at least a portion of the proximal portion 510 of the implant 500. Accordingly, the distal portion 530 of the implant 500 may be releasably coupled with a portion of bone (e.g., screwed in using the handle 760) with the implant then released from the releasable coupling with the engagement feature 808 of the instrument 800.

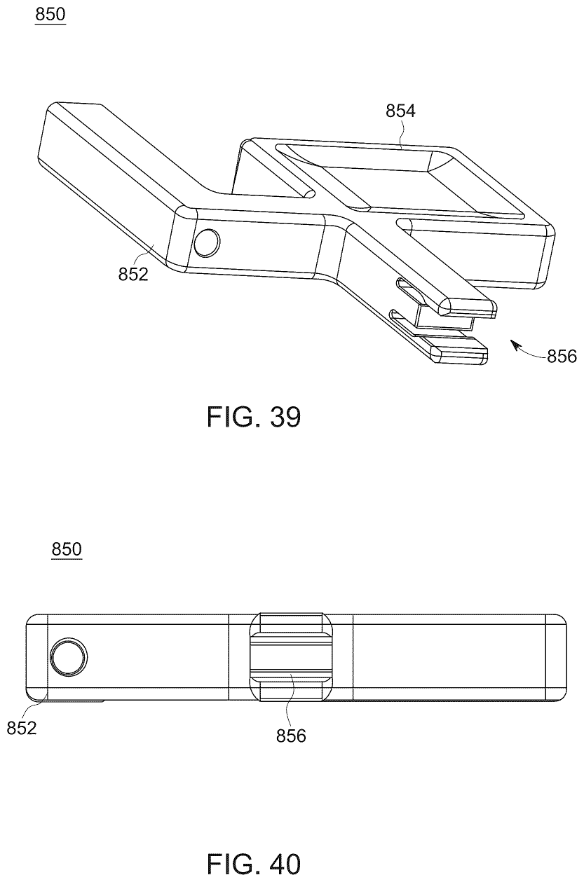

Referring now to FIGS. 38-40, and instrument 850 (e.g., a drill guide, positioning guide, etc.) is shown, according to an exemplary embodiment. The instrument 850 may be configured to be implemented with various systems and/or implants shown and described herein but for the sake of brevity will be described relative to the implant 500. The instrument 850 includes a guide portion 852 as well as a handle 854 having a substantially square/rectangular geometry. The instrument 850 may be gripped and manipulated by a physician via the handle 854 such that the guide portion 852 may be positioned in a desired location. The guide portion 852, which extends from the handle 854, may be configured to receive a stabilization wire (for example, a stabilization wire such as a stabilization wire 960 as shown in FIG. 46), a drill bit, or other similar components. The instrument 850 is also shown to include an engagement feature 856 which also extends from the handle 854. As shown, the engagement feature 856 include a pair of projections defining a recess which may be configured to releasably couple with the plurality of flats 522 of the implant 500. Accordingly, when coupled with the implant 500 the instrument 850 may be positioned using the handle 854 such that the guide portion is in a desired location relative to a bony component for a stabilization wire (or drill bit) may be driven through the guide portion 852 and into a bony fragment. In some aspects, the engagement feature 856 may be spaced longitudinally from the guide portion such that when the engagement feature 856 is releasably coupled with the implant 500, a longitudinal axis of the opening 512 of the implant 500 aligns with a longitudinal axis of the guide portion 852 (e.g., such that a stabilization wire or other component driven through the guide portion and through bone would also pass through the opening 512). Accordingly, the instrument 850 may be configured to manipulate the proximal portion 510 of the implant 500 within an opening (e.g., intramedullary canal) of a bony fragment such that a stabilization wire may be driven through the guide portion 852 and the opening 512 in order to retain the implant 500 in a desired position within the opening of the bony fragment.

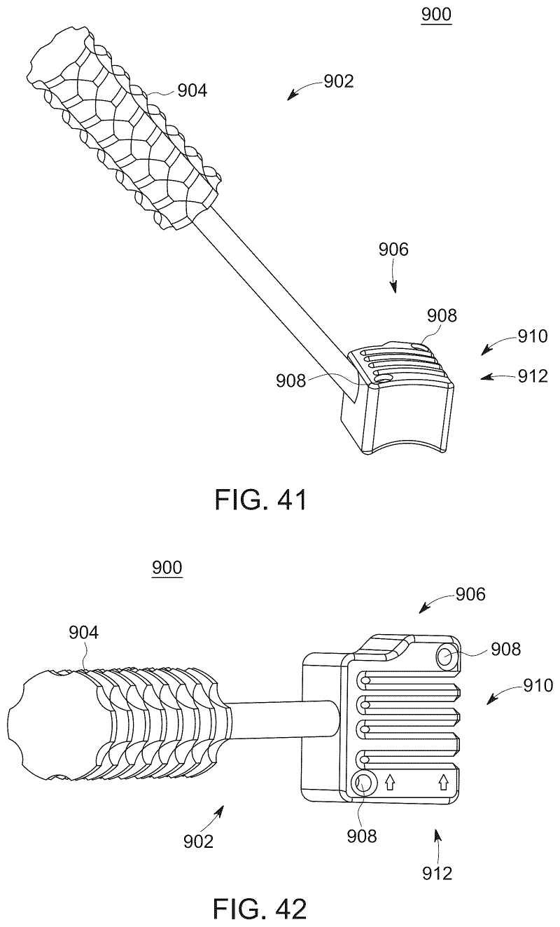

Referring now to FIGS. 41-42, an instrument 900 (e.g., a cut guide) is shown, according to an exemplary embodiment. The instrument 900 may be implemented in conjunction with one or more of the systems, implants, instruments, and/or methods shown and described herein, or may also be implemented in conjunction with other components not shown and described herein. The instrument 900 is shown to include a handle portion 902 having a textured portion 904 disposed on at least a portion thereof, where said texture portion 904 may be configured to aid a physician in gripping and/or manipulating the instrument 900. The instrument 900 is also shown to include a base 906 which may be integral or coupled with a portion of the shaft 902 opposite the textured portion 904. The base 906 is shown to include at least one through hole 908 extending through the base 906 such that a stabilization wire may be placed within the at least one through hole 908 and into a bone disposed below the base 906 so as to releasably couple the base 906 (and the instrument 900) with a portion of a bone. The base 906 is also shown to include at least one slot 910 extending into a base 906 from an edge thereof (e.g., and open-sided slot). In some aspects, the at least one slot 910 may be configured to receive a saw blade or other cutting instrument therein and therethrough to guide one or more cuts to a bone with which the instrument 900 is coupled. In some aspects, the base 906 may also include various markings on surfaces thereof, for example to indicate size or part number, identification of the at least one slot 910, and or other information.

Referring now to FIGS. 43-44, an instrument 920 is shown, according to an exemplary embodiment. Similar to other instruments shown and described herein, the instrument 920 may be implemented in conjunction with one or more of the systems, implants, instruments, and/or methods also shown and described herein, or may also be implemented in conjunction with other components not shown and described herein. The instrument 920 is shown to include a shaft portion 922 having a substantially rectangular shape with a texture 924 disposed on at least a portion of the shaft portion 922. The shaft portion 922 is also shown to include at least one window 926 disposed substantially adjacent the texture 924. In some aspects, the shaft portion 922 includes a contour 928 disposed opposite the shaft portion 922 from the texture 924. Collectively, the texture 924 and the contour 928 may aid a physician in gripping and/or manipulating the instrument 920. In some aspects, the shaft portion 922 may include one or more markings 930 disposed thereon indicating size, product information, or other information. The instrument 920 also includes a base portion 932 extending at a substantially oblique angle from the shaft portion 922 adjacent the texture 924. The base portion 932 is shown to have a lesser lateral dimension than the shaft portion 922. The base portion 932 is shown to include a projection 934 which, as shown, has a substantially tapered cylindrical geometry and is positioned such that a longitudinal axis of the projection 934 extends in a plane substantially perpendicular to that in which the surface of the base portion 932 is disposed. Further, the projection 934 is shown to extend in substantially the direction of the shaft portion 922. The projection 934 may be configured to punch an opening (e.g., open or expand a volume in bone such as an intramedullary canal) that will subsequently be occupied by a portion of one of the implants as shown and described herein.

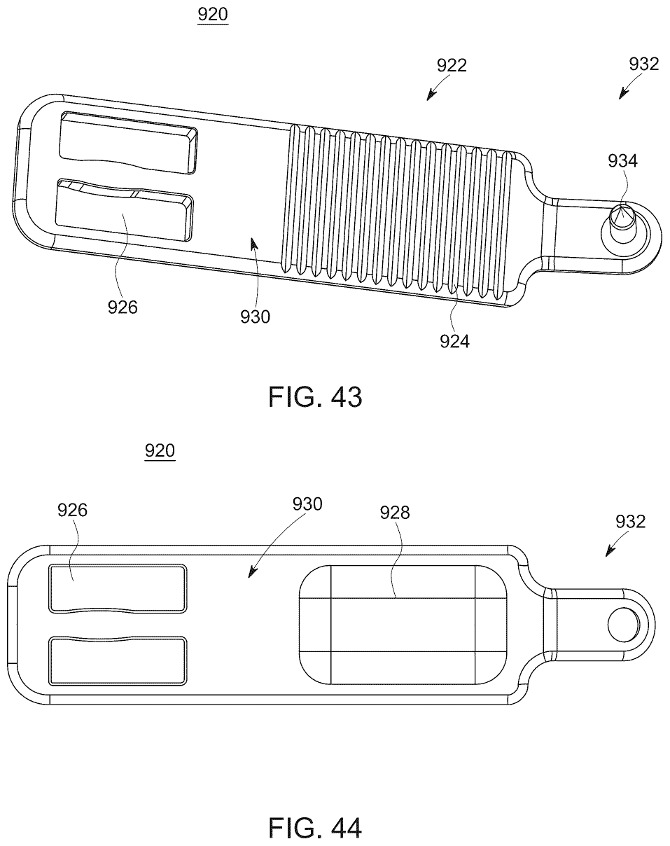

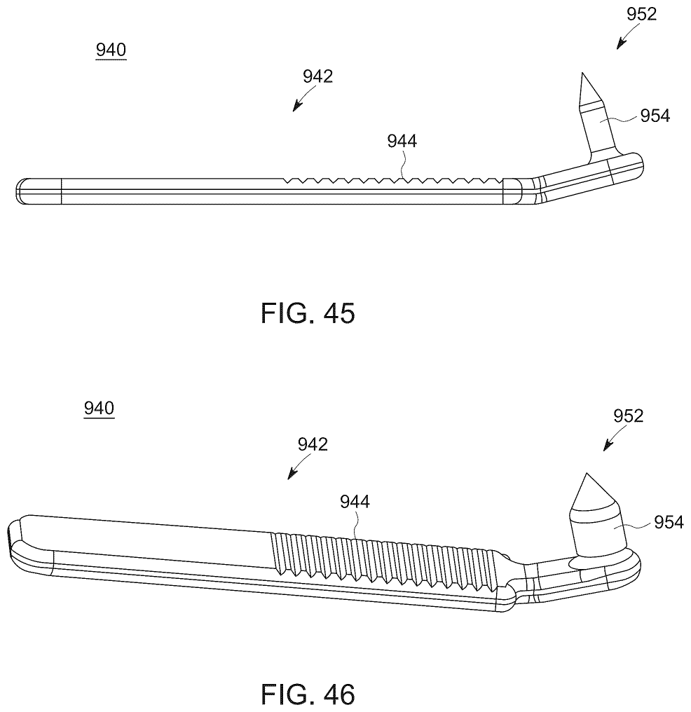

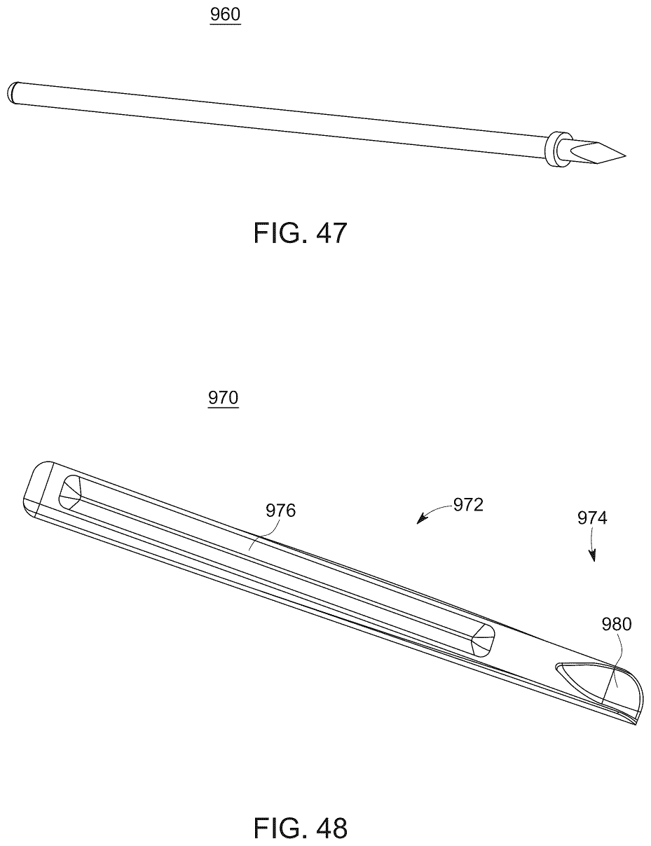

Referring now to FIGS. 45-46, an instrument 940 is shown, according to an exemplary embodiment. Similar to other instruments shown and described herein, the instrument 940 may be implemented in conjunction with one or more of the systems, implants, instruments, and/or methods also shown and described herein, or may also be implemented in conjunction with other components not shown and described herein. The instrument 940 is shown to include a shaft portion 942 having a substantially rectangular shape with a texture 944 disposed on at least a portion of the shaft portion 942. In some aspects, the shaft portion 942 includes a contour disposed opposite the shaft portion 922 from the texture 944. Collectively, the texture 944 and the contour may aid a physician in gripping and/or manipulating the instrument 940. The instrument 940 also includes a base portion 952 extending at a substantially oblique angle from the shaft portion 942 adjacent the texture 944. The base portion 952 is shown to have a lesser lateral dimension than the shaft portion 942. The base portion 952 is shown to include a projection 954 which, as shown, has a substantially tapered cylindrical geometry and is positioned such that a longitudinal axis of the projection 954 extends in a plane substantially perpendicular to that in which the surface of the base portion 952 is disposed. Further, the projection 954 is shown to extend in substantially the direction of the shaft portion 952. The projection 954 may be configured to punch an opening (e.g., open or expand a volume in bone such as an intramedullary canal) that will subsequently be occupied by a portion of one of the implants as shown and described herein. In some aspects, the projection 954 may be substantially larger in height and/or lateral dimension than the projection 934 of the instrument 920. Further, in some aspects a system may be provided with the instruments 920. and 940, as well as other instruments with various components of each being various sizes.

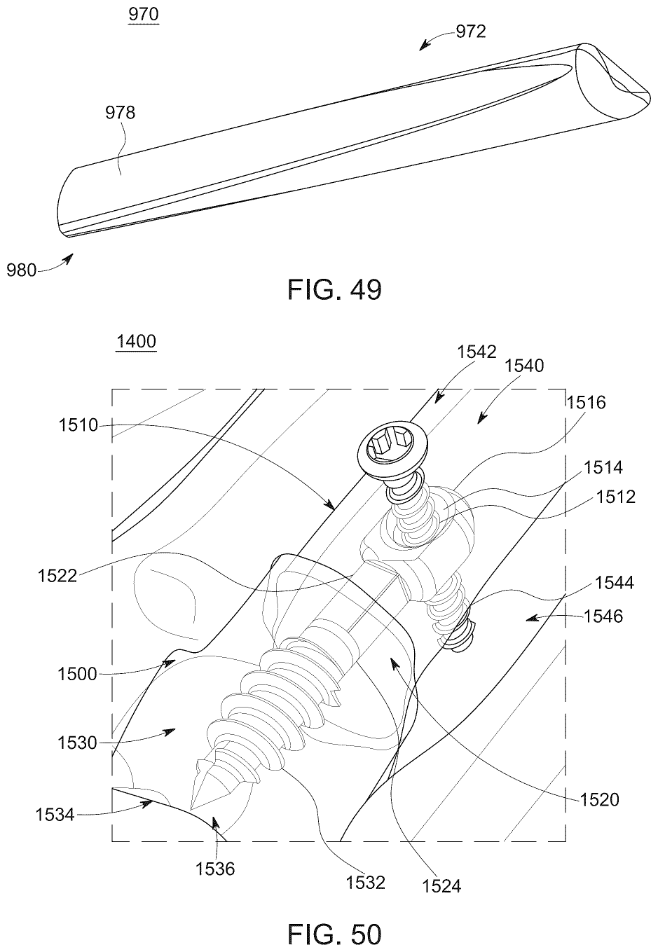

Referring to FIGS. 48-49, an instrument 970 is shown, according to an exemplary embodiment. Similar to other instruments shown and described herein, the instrument 970 may be implemented in conjunction with one or more of the systems, implants, instruments, and/or methods also shown and described herein, or may also be implemented in conjunction with other components not shown and described herein. The instrument 970 is shown to include a shaft portion 972 as well as a base portion 974, where the shaft and base portions 972, 974 are integral with one another. The shaft portion 972 is shown to include a concavity 976 (e.g., a depression, etc.) in a top surface thereof, where said concavity 976 has a substantially u-shaped or v-shaped volume extending along a majority of the length of the shaft portion 972. The shaft portion 972 may also include a similar geometry (e.g., a u-shape or v-shape) which may include one or more contours which, in conjunction with the concavity 976, may be configured to aide a physician in manipulating the instrument 970. The instrument 970 is further shown to include a recess 980 disposed at an end portion of the base portion 974. In some aspects, the recess 980 may be open-ended and extend a volume into the base portion 974 from an outer edge of the base portion 974. Accordingly, the recess 980 may be configured to accommodate at least a portion of an implant including but not limited to those shown and described herein and facilitate manipulation of said implant during an implantation process/procedure.

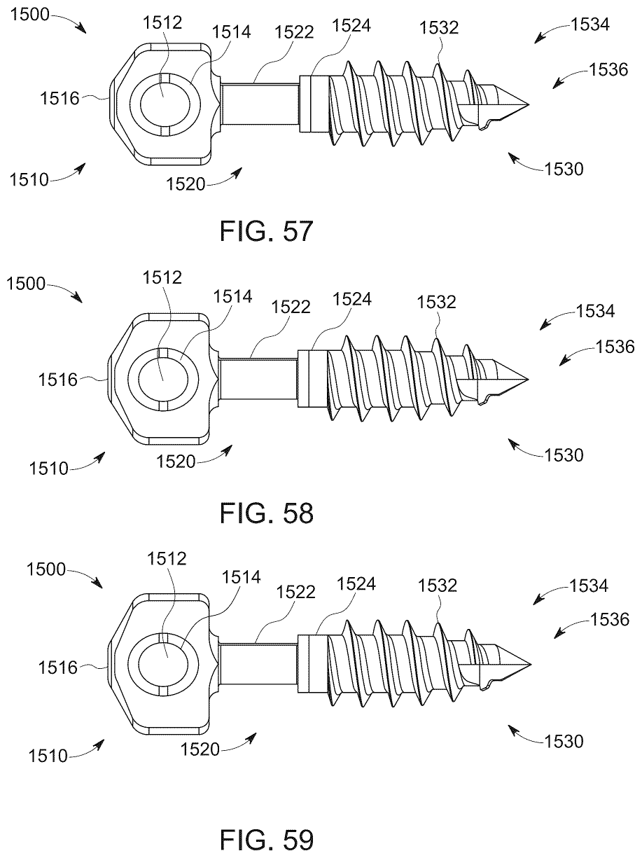

Referring now to FIGS. 50-61, an implant system 1400 and components thereof that may be implemented therewith are shown, according to an exemplary embodiment. The implant system 1400 is shown to include an implant 1500 as well as a screw (e.g., a fastener, etc.) 1540. As shown, the screw 1540 includes a head 1542 as well as a threading 1544 extending along a length of the screw shaft and terminating at a tip 1546, with the tip 1546 positioned substantially opposite the threading 1544 from the head 1542. As shown in FIG. 50, at least a portion of the screw 1540 (e.g., the threading 1544 and the portion of the screw 1540 on which the threading 1540 is disposed on the outer surface thereof) is configured to releasably couple the implant 1500 with the screw 1540. Further, the screw 1540 may be configured to couple with the implant 1500 via one or more bones of portions of bone of a patient so as to provide compression of two bony components or segments toward one another. For example, a first portion of the implant 1500 may be coupled with a first bony portion, and a second portion of the implant1 500 may be coupled with a second bony portion via the screw 1540 (which is thus coupled with both the implant and the second bony portion) so as to provide compression between the first and second bony portions via the implant 1500 and the screw 1540.

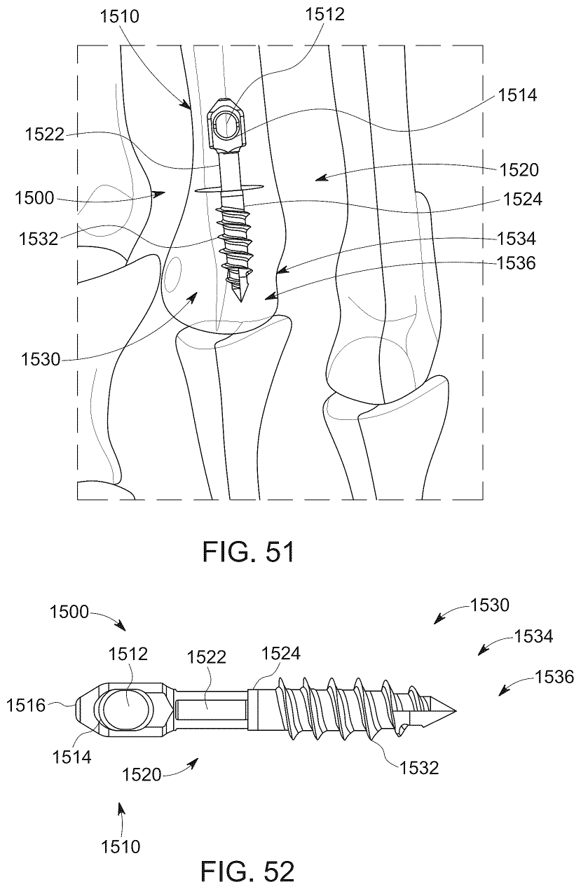

The implant 1500 is shown to include a proximal portion 1510, a distal portion 1530, and a central portion 1520 disposed between the proximal portion 1510 and the distal portion 1530. The notation of proximal and distal portions 1510 and 1530, respectively, is arbitrary as shown in FIGS. 50-51 (and similar to that shown in FIGS. 18-24). However, when implanted in various portions of the human anatomy, either the proximal portion 1510 or the distal portion 1530 may be positioned proximally (and with the other portion positioned distally) relative to the anatomy. As shown, the proximal, central, and distal portions 1510, 1520, and 1530 are substantially centered about a common longitudinal axis. In some aspects, one or more of the proximal, central, and distal portions 1510, 1520, 1530 may include the same and/or similar geometric shapes and/or cross-sections, for example a substantially cylindrical cross-section. As shown, each of the proximal, central, and distal portions 1510, 1520, and 1530 are integral with one another. However, in some aspects one or more of the proximal, central, and distal portions 1510, 1520, and 1530 may be releasably couplable with one another and/or with other components of an implant system.

The proximal portion 1510 of the implant 1500 is shown to extend from the central portion 1520 in a direction substantially opposite from the distal portion 1530. As shown, the proximal portion 1510 includes a substantially greater lateral dimension than that of the central portion 1520 in at least one direction (e.g., wider in at least a medial, lateral, anterior and/or posterior direction with said directions assigned arbitrarily; or as shown “wider” in left and right/horizontal directions and/or “taller” in top/bottom directions). The proximal portion is shown to include an opening 1512 shown as a through hole centrally positioned in the proximal portion 1510 and extending from one surface (e.g., a top surface) through the proximal portion 1510 to a second opposite surface (e.g., a bottom surface). As shown, the opening 1512 is positioned such that a longitudinal axis of the opening 1512 (e.g., an axis about which the opening 1512, which is shown as a circular/cylindrical opening, but may include other geometries in alternate embodiments, is centered concentrically about) would be positioned substantially perpendicular (e.g., in perpendicular planes) relative to the longitudinal axis of the implant 1500. In some aspects, the longitudinal axis of the opening 1512 may intersect with the longitudinal axis of the implant 1500. The opening 1512 is configured to receive at least a portion of a fastener, for example the screw 1540, therethrough such that at least a portion of the screw 1540 abuts a portion of the proximal portion 1510 that defines a lateral dimension of the opening 1512 (e.g., contacts the threading/shaft portion of the screw 540 as shown).

The proximal portion 1510 is further shown to include a tapered edge 1514 configured adjacent the opening 1512. As shown, the entrance to the opening 1512 relative to a top or bottom surface (e.g., a surface in a plane perpendicular to the longitudinal axis of the opening 1512) of the proximal portion 1510 is shown to have a tapered edge 1514 (e.g., a graduated, rounded, or otherwise non-perpendicular interface between the top/bottom surfaces of the proximal portion 1512 and the surfaces of the proximal portion 1510 that laterally define the opening 1512. The proximal portion 1510 is also shown to include a tip 1516 arranged at an end of the proximal portion 1510 opposite that of the interface with the central portion 1520. The tip 1516 is shown to have a tapered geometry where the lateral dimension of the proximal portion 1510 at the tip 1516 is lesser than that of the proximal portion directly adjacent the opening 1512 or the interface with the central portion 1520.

The central portion 1520 of the implant 1500 is shown to have a substantially rectanguloid geometry extending from an end of the proximal portion 1510 to a proximal-most portion of the distal portion 1530 of the implant 1500. As shown, the central portion 1520 has a lesser longitudinal dimension than that of the proximal portion 1510. However, in alternate embodiments, the central portion 1520 may include a greater longitudinal dimension than shown in FIGS. 50-59. Conversely, in some embodiments the central portion 1520 may include a lesser longitudinal dimension than shown or, in some aspects, may be absent from the implant 1500 (e.g., the proximal and distal portions 1510, 1530 are integral with one another). The central portion 1520 is shown to include a plurality of flats 1522 (shown as four flats) arranged about the central portion 1520 (e.g., outer surfaces of the rectanguloid). In some aspects, the flats 1522 may be configured to interface with or facilitate releasable coupling with one or more instruments incorporated in conjunction with the system 1400. Further, in some embodiments the central portion 1520 may include alternate geometries to the rectanguloid shown, in which case the central portion 1520 may include a lesser or greater number of flats 1522 than shown (or such an embodiment may also include four flats as shown).

The central portion 1520 is further shown to include a cylindrical portion 1524 arranged between the plurality of flats 1522 and the distal portion 1530. In some aspects, the plurality of flats 1522 may include a tapered transition into the cylindrical portion 1524. The central portion 1520 may also include a marking arranged on an outer surface thereof. Such a marking may span the outer dimension of the central portion 1520 and may function as a depth indicator to aid in the insertion of the implant 1500. In some aspects, such a marking may include multiple markings, for example multiple rings indicating various depths of insertion when the implant 1500 is implanted.

The distal portion 1530 is shown to include a substantially cylindrical geometry adjacent the central portion 1520, with the geometry tapering toward the end of the distal portion 1530 opposite the central portion 1520. The distal portion is further shown to include a threading 1532 extending circumferentially (e.g., as a spiral configuration) from a portion adjacent the central portion 1520 to a tip 1534 at the terminal end of the distal portion 1530. In some aspects, the threading 1532 may be configured to be oversized relative to the circumference/taper of the tapered cylindrical geometry of the distal portion 1530. The tip 1534 may include a tap 1536 (e.g., a self-tapping portion, cutting flute or flutes, etc.) configured to facilitate implantation within an intramedullary canal of a bone (e.g., so as to eliminate the need for a pilot hole or facilitate the implantation of the distal portion 1530 of the implant 1500 after an opening in an intramedullary canal is punched rather than drilled).

The system 1400 is configured such that the distal portion 1530 may be rotatably coupled (via the threading 1532) with a first bony fragment (e.g., an intramedullary canal which may be manipulated or “punched” to create a volume sufficient to receive and couple with the distal portion 1530) such as a distal portion of a metatarsal. The proximal portion 1510 is similarly configured to couple with a second bony fragment (e.g., an intramedullary canal which may be manipulated or “punched” to create a volume sufficient to receive and couple with the proximal portion 1510) where the proximal portion is positioned such that a longitudinal axis of the opening 1512 extends substantially perpendicular to a longitudinal axis of an anatomical opening (e.g., an intramedullary canal). The proximal portion 1510 may be disposed in such an anatomical opening such that the screw 1540 may be inserted along the longitudinal axis of the opening 1512 (e.g., in a plane perpendicular to the longitudinal axis of the implant 1500) such that a portion of the threading 1544 of the screw 1540 contacts a proximal most edge of the portion of the proximal portion 1510 that defines the proximal-most portion of the opening 1512. Accordingly, such positioning of the screw 1540 biases the implant 1500 in a proximal direction (relative to the implant 1500) thus compressing the first and second bony fragments.

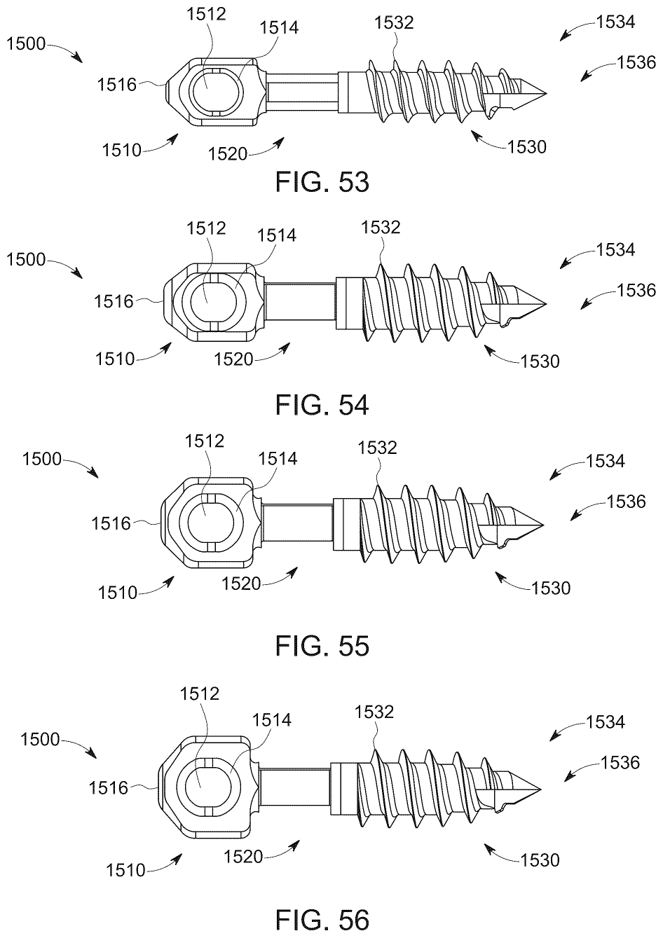

The implants 1500 as shown in FIGS. 50-59 may have varying dimensions of the proximal portion 1510. For example, the implant 1500 of FIG. 52 includes a lesser lateral dimension of the proximal portion 1510 than that of the implant 1500 of FIGS. 53-59, with the implant 1500 of FIG. 59 having the greatest lateral dimension of the proximal portion 1510. In some aspects, the lateral dimension (in at least one plane) of the proximal portion 1510 of the implant 1500 may be lesser than (e.g., FIG. 52), substantially equal to (e.g., FIG. 53), or greater than (e.g., FIG. 59) the lateral dimension of the outermost portion of the threading 1532.

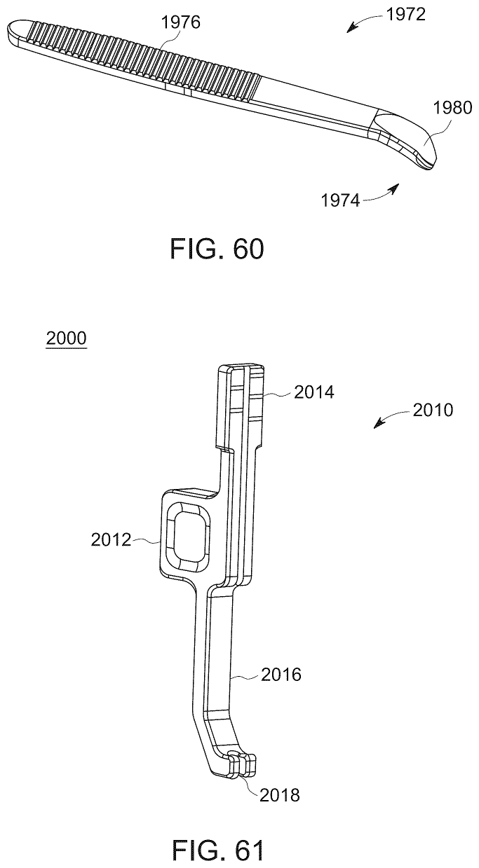

Referring now to FIG. 60, an instrument 1970 is shown, according to an exemplary embodiment. Similar to other instruments shown and described herein, the instrument 1970 may be implemented in conjunction with one or more of the systems, implants, instruments, and/or methods also shown and described herein, or may also be implemented in conjunction with other components not shown and described herein. The instrument 1970 is shown to include a shaft portion 1972 as well as a base portion 1974, where the shaft and base portions 1972, 1974 are integral with one another. The base portion 1974 is configured at an oblique angle (e.g., approximately between 20 and 70 degrees) relative to a longitudinal axis of the shaft portion 1972. The shaft portion 1972 is shown to include an ergonomic feature 1976 (e.g., a texture, surface coating, ridges, etc.) on a top surface thereof, and extending along a majority of the length of the shaft portion 1972. The shaft portion 1972 may also include an alternate geometry (e.g., a u-shape or v-shape) which may include one or more contours which, in conjunction with the ergonomic feature 1976, may be configured to aide a physician in manipulating the instrument 1970. The instrument 1970 is further shown to include a recess 1980 disposed at an end portion of the base portion 1974. In some aspects, the recess 1980 may be open-ended and extend a length and depth into the base portion 1974 from an outer edge of the base portion 1974. Accordingly, the recess 1980 may be configured to accommodate at least a portion of an implant including but not limited to those shown (e.g., 1500 and 1540) and described herein and facilitate manipulation of the implant during an implantation process/procedure.

Referring now to FIG. 61, an instrument 2000 is shown, according to an exemplary embodiment. The instrument 2000 may be implemented in conjunction with one or more implants, implant systems, and/or instruments shown and described herein previously. The instrument 2000 includes an upper portion 2010, which is integral with a lower portion 2016 where both the upper and lower portions 2010, 2016 are substantially linear and, as shown, are positioned off-axis from one another (e.g., the two portions do not share a common longitudinal axis). The upper portion 2010 includes a protrusion 2012 extending laterally therefrom and having a substantially rectangular (e.g., square) geometry. As shown the protrusion 2012 includes a depression configured to facilitate gripping of the instrument 2000 by the protrusion 2012 by a physician intraoperatively. The lower portion 2016 is shown to extend downward from a side of the protrusion 2012 (e.g., a side 90-degrees from the side which abuts the upper portion 2010) and includes a coupling portion 2018 at a distal-most portion thereof. The coupling portion 2018 is shown to extend from the lower portion 2016 at an oblique angle and is configured to receive at least a portion of an instrument, fastener, or other component at least partially therein. In some aspects, one or more fasteners (e.g., screws) and/or instruments shown and described previously herein may be implemented in conjunction with the instrument 2000 in accordance with performing a surgical procedure. The instrument 2000 also includes depth markings 2014 to facilitate depth measurement and fastener selection intraoperatively.

In an exemplary surgical method, one or more of the implants/implant systems and instruments shown and described herein, as well as other instruments common to orthopedic surgical procedures, may be implemented. For example, the implant 1500 may be used (e.g., implanted) in accordance with a physician performing a procedure on a metatarsal of a patient (for example, a metatarsal shortening procedure). In such a procedure, a physician may perform an osteotomy to the metatarsal (which may be guided by a cut guide the same as and/or similar to that shown and described previously herein) in a plane substantially orthogonal to a longitudinal axis (e.g., long axis) of the metatarsal so as to separate the metatarsal into two bony fragments (referred to hereinafter as proximal and distal bony fragments). One or more implant trials may then be subsequently placed into the intramedullary canal of the proximal bony fragment, beginning with the smallest trial provided in a set (e.g., smallest diameter) and progressing to trials with increasing size until a trial fits snugly in the intramedullary canal and the next largest size does not fit snugly. For some patients, the smallest trial my not fit in the intramedullary canal and a reamer may be used to expand the diameter of the intramedullary canal to accommodate the smallest implant trial.

The instrument 1970 may then be implemented to manipulate the distal bony fragment such that the surface created by the osteotomy cut is exposed. A pilot hole may then be drilled perpendicular to the osteotomy cut plane into the distal fragment using an instrument with a mechanical depth stop (e.g., an olive wire, etc.). The implant corresponding to the predetermined appropriate trial size, for example the implant 1500, may then be coupled with an insertion instrument (e.g., the instrument 780 as shown in FIG. 37, which may be adapted to accommodate the implant 1500 or other implants) to facilitate insertion of the distal portion 1530 and threading 1532 into the distal bony fragment of the metatarsal by manipulating the insertion instrument in a clockwise direction until the distal-most portion of the insertion instrument contacts the distal bony fragment of the metatarsal. At least a portion of the proximal portion 1510 of the implant 1500 may then be inserted in the intramedullary canal of the proximal bony fragment of the first metatarsal.

The instrument 850 may then be releasably coupled with a portion of the implant 1500 engaged with the engagement feature 856 such that the guide portion 852 (and the bore extending therethrough) are positioned superior relative to the proximal bony fragment. The proximal and distal bony fragments are then compressed with the instrument 850 as least partially disposed between the bony fragments. A k-wire or other similar instrument may then be placed and subsequently removed through the bore of the guide portion 852 and into the first metatarsal, with the instrument 2000 used to measure the depth of the k-wire and aid a physician in selecting a fastener with a length corresponding to the depth indicated by the instrument 2000. The instrument 850 may then be decoupled from the implant 1500, with the proximal and distal bony fragments compressed such that the osteotomy sites abut one another. Such compression biases the opening 1512 of the implant 1500 to a coaxial position with the bore created by the k-wire. The selected fastener/screw is then placed through the bore in the metatarsal such that at least a portion of the fastener/screw is received through the opening 1512 (and potentially contacts a proximal side of the opening) so as to retain the implant within the proximal bony fragment and provide compression between the two bony fragments.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

What is claimed is:

1. An implant, comprising:
a proximal portion;
a distal portion; and
a central portion disposed between the proximal portion and the distal portion,
wherein the implant further comprises a longitudinal axis and the proximal portion, the distal portion and the central portion are centered along the longitudinal axis.

2. The implant of claim 1, wherein the proximal portion, the distal portion and the central portion are integral to each other.

3. The implant of claim 1, wherein the proximal portion, the distal portion and the central portion are three distinct portions and are couplable to each other.

4. The implant of claim 1, wherein the proximal portion comprises a top surface and a bottom surface with the top surface and bottom surface being planar and parallel to each other.

5. The implant of claim 4, wherein the proximal portion further comprises an opening extending from the top surface to the bottom surface, wherein the opening is sized to receive a bone fastener.

6. The implant of claim 5, wherein the opening has a central axis, wherein the central axis is perpendicular to the longitudinal axis of the implant.

7. The implant of claim 5, wherein the opening further comprises at least one of a tapered, a rounded or a gradient surface that extends from the top surface to an inside wall of the opening, and wherein the opening further comprises at least one of a tapered, a rounded or a gradient surface that extends from the bottom surface to the inside wall of the opening, wherein the at least one of a tapered, a rounded or a gradient surfaces facilitate polyaxial movement of the bone fastener when inserted into the opening of the implant.

8. The implant of claim 1, wherein the proximal portion further comprises a lateral dimension and a longitudinal dimension, wherein the lateral dimension is greater than the longitudinal dimension.

9. The implant of claim 1, wherein the proximal portion further comprises a lateral dimension and a longitudinal dimension, wherein the lateral dimension is less than the longitudinal dimension.

10. The implant of claim 1, wherein the proximal portion further comprises a lateral dimension and a longitudinal dimension, wherein the lateral dimension is equal to the longitudinal dimension.

11. The implant of claim 1, wherein the proximal portion further comprises a tip section, wherein the tip section comprises a planar surface proximate to two tapered surfaces, wherein the planar surface length is less than the lateral dimension of the proximal portion proximate to the opening.

12. The implant of claim 1, wherein the central portion has a longitudinal dimension, wherein the longitudinal dimension of the central portion is less than the longitudinal dimension of the proximal portion.

13. The implant of claim 1, wherein the central portion has a longitudinal dimension, wherein the longitudinal dimension of the central portion is greater than the longitudinal dimension of the proximal portion.

14. The implant of claim 1, wherein the central portion comprises a substantially rectanguloid section and a cylindrical section.

15. The implant of claim 14, wherein the substantially rectanguloid section extends from a distal end of the proximal portion to a proximal end of the distal portion.

16. The implant of claim 14, wherein the substantially rectanguloid section comprises a circumferential outer surface, wherein the circumferential outer surface comprises at least one flat surface disposed thereon.

17. The implant of claim 16, wherein the at least one flat surface is configured to facilitate the coupling of an insertion instrument.

18. The implant of claim 16, wherein the at least one flat surface extends for the longitudinal dimension of the substantially rectanguloid section.

19. The implant of claim 14, wherein the cylindrical section of the central portion extends distally from the substantially rectanguloid section and tapers into the proximal end of the distal portion of the implant.

20. The implant of claim 16, wherein the circumferential outer surface further comprises at least one marking thereon, wherein the at least one marking designates a depth of insertion of the implant into a bone.

21. The implant of claim 1, wherein the distal portion comprises a threading extending along at least a portion of the distal portion and a tip positioned at a distal end of the distal portion.

22. The implant of claim 21, wherein the lateral dimension of the proximal portion may be at least one of greater to, substantially equal to or less than an outermost dimension of the threading.

23. The implant of claim 1, wherein the distal portion tapers from its proximal end to the distal end.

24. The implant of claim 21, wherein the tip comprises at least one of a self-tapping mechanism or at least one cutting flute.

25. The implant of claim 21, wherein the threading is circumferential disposed spiral threads.

26. An implant system, comprising:
an implant for insertion into a first bone comprising:
a proximal portion, comprising:
an opening extending through the proximal portion of the implant;
a distal portion, comprising:
a threading extending along at least a portion of the distal portion;
a tip positioned at an end of the distal portion opposite the proximal portion; and
a tapping feature positioned at the tip of the distal portion;
a central portion disposed between the proximal portion and the distal portion and comprising at least one flat disposed about an outer surface thereof; and
a fastener, wherein the fastener is sized to be inserted into and through the opening and into a second bone.
